(19) United States
(12) Patent Application Publication
(10) Pub. No.: US 2009/0156895 A1
(43) Pub. Date: Jun. 18, 2009

(54) PRECISE ENDOSCOPIC PLANNING AND VISUALIZATION

(75) Inventors: William E. Higgins, State College, PA (US); Jason D. Gibbs, State College, PA (US)

Correspondence Address: GIFFORD, KRASS, SPRINKLE,ANDERSON & CITKOWSKI, P.C, PO BOX 7021, TROY, MI, 48007-7021, US

(73) Assignee: The Penn State Research Foundation, University Park, PA (US)

(21) Appl. No.: 12/344,040

(22) Filed: Dec. 24, 2008

Related U.S. Application Data
(63) Continuation-in-part of application No. 12/018,953, filed on Jan. 24, 2008.
(60) Provisional application No. 60/887,472, filed on Jan. 31, 2007.

(51) Int. Cl. A61B 1/012

(52) U.S. Cl. 600/104; 600/117

(57) ABSTRACT

Endoscopic poses are used to indicate the exact location and direction in which a physician must orient the endoscope to sample a region of interest (ROI) in an airway tree or other luminal structure. Using a patient-specific model of the anatomy derived from a 3D MDCT image, poses are chosen to be realizable given the physical characteristics of the endoscope and the relative geometry of the patient's airways and the ROI. To help ensure the safety of the patient, the calculations also account for obstacles such as the aorta and pulmonary arteries, precluding the puncture of these sensitive blood vessels. A real-time visualization system conveys the calculated pose orientation and the quality of any arbitrary bronchoscopic pose orientation. A suggested pose orientation is represented as an icon within a virtual rendering of the patient's airway tree or other structure. The location and orientation of the icon indicates the suggested pose orientation to which the physician should align during the procedure.

### REFERENCE TO RELATED APPLICATIONS

This application is a continuation-in-part of U.S. patent application Ser. No. 12/018,953, filed Jan. 24, 2008, which claims priority from U.S. Provisional Patent Application Ser. No. 60/887,472, filed Jan. 31, 2007, the entire content of both of which is incorporated herein by reference.

### GOVERNMENT SPONSORSHIP

This work was partially supported by Grant Nos. CA074325 and CA091534 from the National Cancer Institute of the NIH NIBIB Grant No. EB000305. The U.S. Government may have rights in this invention.

### FIELD OF THE INVENTION

This invention relates generally to endoscopic planning and visualization and, in particular, to methods and apparatus facilitating the precise determination of an optimal endoscopic configuration for sampling a region of interest (ROI).

### BACKGROUND OF THE INVENTION

Co-pending U.S. patent application Ser. No. 12/018,953, entitled “Methods and Apparatus for 3D Route Planning Through Hollow Organs” describes automatically computing appropriate routes through hollow branching organs from volumetric medical images for image-based reporting and follow-on medical procedures. While these methods are applicable to a wide range of circumstances, the motivation was the diagnosis and treatment of lung cancer. The state-of-the-art process for lung cancer assessment begins with the acquisition of a multidetector computed tomography (MDCT) scan of the patient's chest. In this three dimensional (3D) image, the physician looks for the presence of suspicious growths [14, 15]. If a suspicious growth is found, the physician may then choose to perform a minimally-invasive procedure known as bronchoscopy [15, 28, 32, 34].

In a bronchoscopic procedure, the physician inserts a long, thin, flexible videobronchoscope into the patient's airway tree and maneuvers the instrument to sample suspect lesions, or a diagnostic region of interest (ROI), as observed in the 3D MDCT image [34, 32, 1, 15, 30, 31, 2]. A camera and light source at the tip of the bronchoscope allow the bronchoscopist to see the internal structures of the patient's airway tree. Using this feedback, the bronchoscopist navigates to an appropriate sample location. At the sample location, the physician inserts a bronchoscopic accessory, such as a needle, forceps, or diagnostic brush, through the hollow working channel of the bronchoscope to sample the lesion [34].

However, physicians often get disoriented in the complex branching airway tree. Furthermore, ROIs are often located beyond the airway walls and therefore outside the view of the bronchoscopic camera. For these reasons, bronchoscopies are difficult, error-prone procedures [5, 3, 22, 11, 25, 21]. To help address these issues, the previously filed application Ser. No. 12/018,953 discloses methods for automatically computing appropriate endoscopic routes to ROIs. These automatically-computed routes may be incorporated into a live guidance system to direct physicians through the airways to an appropriate region for sampling the ROI [23, 22, 8, 7, 10].

### SUMMARY OF THE INVENTION

The instant invention extends and improves upon endoscopic planning and visualization in several ways. One enhancement involves the precise determination of an optimal endoscopic configuration for sampling a region of interest (ROI). In one application, apparatus and methods are disclosed for computing a more precise bronchoscopic sampling configuration as opposed to being directed along bronchoscopic routes to a general location for bronchoscopic biopsy. More particularly, instead of indicating a general region for sampling an ROI, a bronchoscopic pose is presented to indicate an optimum or first location and direction for the physician to orient the bronchoscope and to sample the ROI.

In determining a first pose, optimum pose, or a best pose(s), the physician is directed to bronchoscopic configurations that maximize the core sample of the ROI, namely, the size or depth of the core sample. Using a patient-specific model of the anatomy derived from a 3D image such as, for example, a 3D MDCT image, bronchoscopic poses are chosen to be realizable given the physical characteristics of the bronchoscope and the relative geometry of the patient's airways and the ROI. To help ensure the safety of the patient, in one embodiment, calculations account for obstacles such as the aorta and pulmonary arteries, precluding the puncture of these sensitive blood vessels. In another embodiment, a real-time visualization system conveys the calculated pose orientation and the quality of any arbitrary bronchoscopic pose orientation. In this system, a suggested pose orientation is represented as an arrow within a virtual bronchoscopic (VB) rendering of the patient's airway tree. The location and orientation of the arrow indicates the suggested pose orientation to which the physician should align during the procedure. In another embodiment, the visualization system shows the ROI, which is differentially colored to indicate the depth of sample of the ROI at a given VB camera location. The ROI, which may be located outside the airway wall, is blended into the scene at varying color intensities, with brighter intensities indicating a larger depth of sample. The physician can freely maneuver within the VB world, with the visual representation of the quality of a bronchoscopic sample at the current VB camera location updating in real time.

In another embodiment, the visualization system depicts obstacles, which can also be located beyond the airway walls. If within a pre-determined safety envelope, the obstacles are depicted in a unique color, informing the physician of poor locations in which to sample the ROI.

### BRIEF DESCRIPTION OF THE DRAWINGS

FIGS. 1A and 1B are illustrations of components of the route data structure. The original tree segmentation I_{S} is given by the smooth boundaries of FIG. 1A. A path extension (100) is contained within the wavy boundary. The paths, branches, and routes are comprised of view sites, represented by circles. ROIs are depicted as hexagons 110. FIG. 1B the route destination (120) is shown in conjunction with a bronchoscope at a pose orientation that faces the ROI.

FIGS. 2A and 2B are illustrations of sampling criteria. FIG. 2A shows needles from two poses “1” and “2”. Needle 1, while intersecting the ROI 122, does not sample as much of the ROI as needle 2. FIG. 2B shows a needle that intersects an obstacle 130. Such a circumstance must be avoided in route calculations.

FIG. 3 is an illustration of relative geometry of airway tree 140, ROI 150, and bronchoscope 160. The bronchoscope tip fits within the airway when oriented toward the ROI at the pose orientation of (a). The pose orientation of (b), however, is infeasible as the bronchoscope tip protrudes from the airway tree.

FIGS. 4A and 4B are illustrations of a pose orientation coordinate system and needle vector respectively. The pose orientation of FIG. 1A shows the alignment of the bronchoscope. A particular needle vector FIG. 4B may not exactly align with the nominal pose orientation direction, due to uncertainty in the needle orientation.

FIG. 5 is an illustration of view site/voxel associated pose locations. The smooth outer curves represent the airway surfaces, the interior grid is the airway-tree segmentation, and the circles 170 represent view sites. The voxels 180 are nearest, and therefore associated with, the view site 190. The centers of the voxels 180 are candidate pose locations associated with the view site 190.

FIG. 6 is an illustration of ROI partitioning via k-means. The triangles show the k-means locations and the square is a voxel in I_{S}, corresponding to pose location. The arrows represent different pose orientations nₚ^{i,j}, j=1, . . . , |K| at a pose location sₚⁱ.

FIG. 7 is an illustration of the expected depth of sample envelope and safety envelope for a pose. A pose at a segmentation voxel (200) is oriented toward a target section (210) of the ROI 220. The first cone 230 is the envelope considered for the expected depth of sample calculations. The second cone 240 is the safety envelope. The expected depth of sample envelope is completely contained within the safety envelope.

FIG. 8 is an illustration of a global rendering of the airway tree (250), aorta (260), and pulmonary artery (270) corresponding to a case 20349.3.9.

FIG. 9 is an illustration of a depth of sample D(s_{N}ⁱ,d_{N}^{ijk},L_{N}) of a single needle. The needle is oriented along direction d_{N}^{ijk} and the outward-facing normals of the two intersected triangles are t₁ and t₂. The shaded area 280 indicates the interior of the ROI volume.

FIG. 10 is an illustration of a pose orientation icon. The arrow 290 shows where the tip of the bronchoscope should be aligned to optimally sample the ROI (case 20349.3.9). The regions 300 depict the virtual airway walls, the second regions 310 show locations where obstacles (in this case, the pulmonary artery or aorta) are nearby and the region 320 shows the target ROT, which is actually located beyond the airway wall.

FIGS. 11A and 11B are illustrations of an overview of the case illustrating mediastinal visualization in case 20349.3.9, ROI 2 FIG. 6). FIG. 11A shows the airway tree (330), ROI (340), aorta (350), and pulmonary artery (360). This view is from behind the patient so that the ROI is not obscured by the obstacles. The close-up view of FIG. 11B shows these same structures, with the suggested pose orientation 370 calculated using a 4.9 mm bronchoscope with a 10 mm rigid tip.

FIG. 12 is an illustration of the endoluminal depth of sample and obstacle visual cues for case 20349.3.9, ROI 2 (FIG. 11). The distances below each view give the remaining distance until the suggested pose orientation, indicated by the 4 mm long blue arrow 380. The views on the top show the new visualization approach, compared with the previous virtual bronchoscopic views on the bottom. In the new views, the values 390 (which may be, e.g., bright red) indicate the pulmonary artery or aorta are within 22.5 mm of the virtual camera. The position of the red in each view shows where the extraluminal obstacles are located. ROI 400 may be shown in green. The shades of green 400, which are first visible in (b), may get brighter as the physician nears the ROI 400, indicating the ROI depth of sample at the current pose orientation.

FIG. 13 is an illustration of the effect of ROI section geometry on rendered brightness. The arrows represent a ray along which a single pixel's brightness is calculated. The pixel in (a) will appear brighter than (b) as the ray of (a) intersects 6 voxels instead of the 5 of ray (b).

FIG. 14 is another illustration of pose orientation visualization corresponding to Case 20349.3.3 ROI 416 pose orientation. This case is quite safe as almost none of the obstacle 410 is visible. The expected depth of sample is 9.4 mm, so the physician should expect a good sample. The new visualization shows that this sample can be achieved in a large portion of the view.

FIG. 15 is another illustration of pose orientation visualization corresponding to Case 20349.3.3 ROI 420 pose orientation. This ROI 420 is also quite achievable, although care must be taken to avoid the aorta to the left of the view.

FIG. 16 is another illustration of pose orientation visualization corresponding to Case 20349.3.3 ROI 430 pose orientation. This case, while feasible, is difficult. Both the aorta and PA are visible in the view and are near the ROI 430.

FIG. 17 is another illustration of pose orientation visualization corresponding to Case 20349.3.3 ROI 440 pose orientation. This case is also somewhat challenging with both the PA and aorta present. The expected depth of sample is also small (4.1 mm), suggesting that reliably sampling the lesion may be difficult.

FIG. 18 is another illustration of pose orientation visualization corresponding to Case 20349.3.3 ROI 450 pose orientation. This lesion is straightforward to sample. The pose is safe with an expected depth of sample of 14.9 mm and, as illustrated by shaded area 450 which encompasses nearly the entire view and may be shown as bright green, the physician can be expected to sample this lesion.

FIG. 19 is another illustration of pose orientation visualization corresponding to Case 20349.3.6 ROI 460 pose orientation. The physician needs to be careful to avoid the obstacle 462 in this case.

FIG. 20 is another illustration of pose orientation visualization corresponding to Case 20349.3.7 ROI 470 pose orientation. Similar to the previous case, the physician needs to be careful to avoid the obstacle 472.

FIG. 21 is another illustration of pose orientation visualization corresponding to Case 20349.3.9, ROI 480, pose orientation. This is a long, narrow ROI surrounded by nearby obstacles 482 and could be difficult to sample.

FIG. 22 is another illustration of pose orientation visualization corresponding to Case 20349.3.9, ROI 490 pose orientation. This is a wide, flat ROI. The suggested sample location is at the broad side of the ROI, resulting in a relatively shallow depth of sample (3.7 mm).

FIG. 23 is another illustration of pose orientation visualization corresponding to Case 20349.3.9, ROI 500, pose orientation. This ROI 500 is also shallow and flat, but in this case is best approached from the narrow side. As such, the physician must be careful aligning the sample to be sure to hit the target.

FIG. 24 is another illustration of pose orientation visualization corresponding to Case 20349.3.11 pose orientation. This case is straightforward, with minimal obstacle present. The expected depth of sample is 11.8 mm, and it is seen that the majority of the view has a good sample depth.

FIG. 25 is another illustration of pose orientation visualization corresponding to Case 20349.3.16, ROI 520, pose orientation. This case is straightforward, with an expected depth of sample of 10.0 mm.

FIG. 26 is another illustration of pose orientation visualization corresponding to Case 20349.3.16, ROI 530, pose orientation. This case is also straightforward with an even larger expected depth of sample (16.2 mm).

FIG. 27 is another illustration of pose orientation visualization corresponding to Case 20349.3.37 ROI 540 pose orientation. This case is somewhat challenging, as the physician needs to avoid the obstacle 542 in the upper-left.

FIG. 28 is another illustration of pose orientation visualization corresponding to Case 20349.3.39, ROI 550 pose orientation. This case has a large expected depth of sample (14.0 mm) but the physician must avoid the obstacle 552 in the upper-left.

FIG. 29 is another illustration of pose orientation visualization corresponding to Case 20349.3.9, ROI 560 pose orientation. Due to the proximity of the ROI to the aorta and pulmonary artery (collectively obstacles 562), the safety envelope was decreased to 100 safety FOV and 20 mm length. With this less-conservative safety envelope, the physician needs to be especially careful when sampling the lesion.

FIG. 30 is another illustration of pose orientation visualization corresponding to Case 20349.3.29 pose orientation. Because the mass 570 is located deep within the airway tree 572 near small airways 574, a smaller bronchoscope was required. A 2.8 mm diameter, 5 mm rigid tip bronchoscope was used in the calculations. The planning indicates a physician would be unable to reach this lesion with the standard 4.9 mm bronchoscope. In this case, the inventor was present for the bronchoscopic procedure. Despite repeated efforts, the physician could not reliably reach an appropriate pose orientation for this case with the 4.9 mm bronchoscope, confirming our calculations.

FIG. 31 is another illustration of pose orientation visualization corresponding to Case 20349.3.39, ROI 580 pose orientation. Because the ROT 580 is co-mingled with the pulmonary artery, a more aggressive safety envelope is required. A 100 safety FOV and 20 mm length safety envelope was used in the planning for the illustrated route. The physician would need to be very careful when performing this procedure, if she felt it warrants TBNA.

FIG. 32 is another illustration of pose orientation visualization corresponding to Case 20349.3.40 pose orientation. Because the ROT 590 is located deeper within the airway tree, a smaller bronchoscope model was used. The bronchoscope was modeled as 2.8 mm in diameter, with a 5 mm rigid tip. As such, this ROI would not be a good candidate for TBNA.

### DETAILED DESCRIPTION OF THE INVENTION

Methods are described for computing an appropriate route destination. Additionally, a visualization system that presents this information to the physician during bronchoscopic procedures is described.

### Branching Organ and Route Representation

As disclosed in patent application Ser. No. 12/018,953, an input for route planning is a 3D gray-scale medical chest image I containing the organ of interest, typically the airway tree, and region of interest (ROI) [13]. From I, an image-segmentation routine generates a binary 3D image I_{S} that defines the airway tree. From I and I_{S}, a 3D surface representing the interior air/airway wall boundary of the airway tree is extracted. Extraction may be carried out by, for example, methods of Gibbs et al. [8, 7]. The medial axes of I_{S} are extracted using existing methods, following the conventions of Kiraly et al. to represent the centerlines [17].

Collectively, the set of all medial axes comprise a tree, T=(V,B,P), where V={v₁, . . . , v_{L}}, is the set of view sites, B={b₁, . . . , b_{M}} is the set of branches, P={p₁, . . . p_{N}} the set of paths, and L, M, and N are integers ≧1. A view site is parameterized by v=(x,y,z,α,β,γ) where the vector s=(x,y,z) gives the location of the view site and the Euler angles α, β, and γ give the orientation. Alternatively, the Euler angles can be represented by the orthonormal vectors n, r, and u, which define a coordinate system located at s. The vector n is the normal view direction (the direction in which the bronchoscope faces), u is the up direction, and r is orthogonal to both n and u. A branch, b={vₐ, . . . vₜ}, vₐ, . . . , vₗεV, combines connected view sites between the organ's topologically significant points. Topologically significant points include the origin (root) of the organ, points where the organ bifurcates, and the terminating points. A branch must contain two and only two topologically significant points that define the beginning and end of the branch, respectively. A path, p={bₐ, . . . , bₘ}, bₐ, . . . , bₘεB, contains a set of connected branches. Paths must begin at a root branch b₁ and terminate at the ends of I_{S}.

These data structures are augmented with the route data structure r={v_{A}, . . . , v_{D}, p}, with some vεV and others new, which consists of a collection of connected view sites [13]. In one embodiment, this data is supplemented with an optional final bronchoscopic pose orientation p. The final view site along the route, v_{D}, is the route destination, which is located near the ROI.

According to the instant invention, the route information is augmented with a final bronchoscopic pose orientation p={sₚ,nₚ,rₚ,uₚ}. A pose p gives the precise location and orientation of the bronchoscope near v_{D} to get the best ROI sample. Note that sₚ is not constrained to be at a location of any view site, and the orientation at the pose location is typically different than the destination view site's orientation. Methods to calculate p are described herein below.

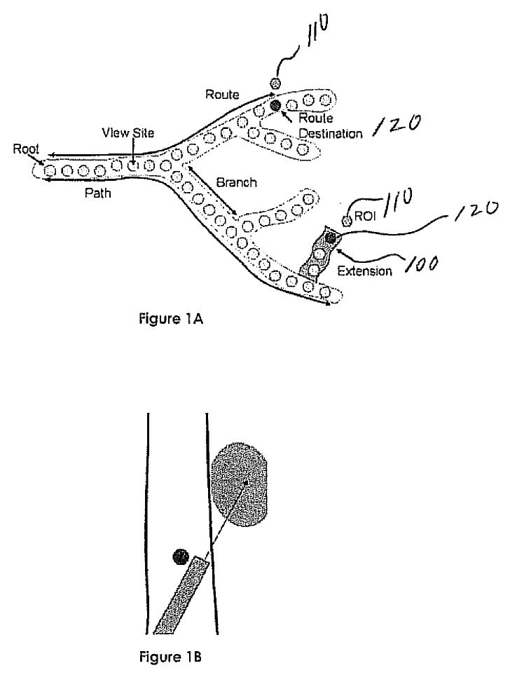

FIGS. 1A and 1B give a visual depiction of these structures. The original organ segmentation is contained within the smooth boundary of FIG. 1A. The medial-axes tree is represented by the circular view sites within the segmentation. ROIs are shown as green hexagons. The route to the ROI at the top of the figure is entirely contained within the original tree. At the bottom of the figure, the route to the ROI requires path extension. FIG. 1B shows an orientation of the bronchoscope near the route destination.

Bronchoscope Pose Orientation

This section describes how to find appropriate bronchoscopic poses for routes that terminate in large airways. First, various factors influencing pose determination are described. To automatically identify the “best” candidate bronchoscopic orientation(s), quantitative scores for potential pose orientations are computed. To determine these pose scores, assumed best routes are those that lead the physician to a bronchoscopic configuration that would maximize the amount of ROI tissue sampled. If the physician uses a needle to sample the ROI, for example, the best orientations are those where the needle takes the largest ROI core samples. FIG. 2A illustrates the scoring of orientations. Pose 1, while sampling the ROI, is not as desirable as Pose 2, which has a greater depth of sample. For brushes and forceps the concept can be similar—the physician wants the sampling device to interact with as many target cells as possible.

FIGS. 2A and 2B are illustrations of sampling criteria. FIG. 2A shows needles from two poses “1” and “2.” Needle 1, while intersecting the ROI, does not sample as much of the ROI as needle 2. FIG. 2B shows a needle that intersects an obstacle 130. Such a circumstance must be avoided in route calculations.

The physical characteristics of the bronchoscope constrain potential configurations. For instance, the airways must be large enough to accommodate the bronchoscope. When sampling lesions outside the airway, the relative geometry between the airway tree, bronchoscope, and ROI must be taken into consideration. An appropriate pose orientation is one in which the bronchoscope can be aligned to face the ROI while still fitting within the airway tree, as illustrated in FIGS. 2A and 2B.

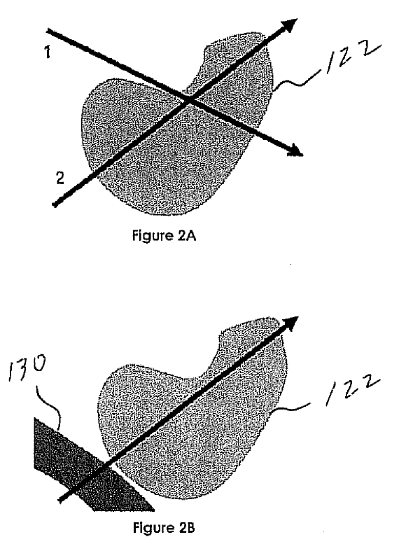

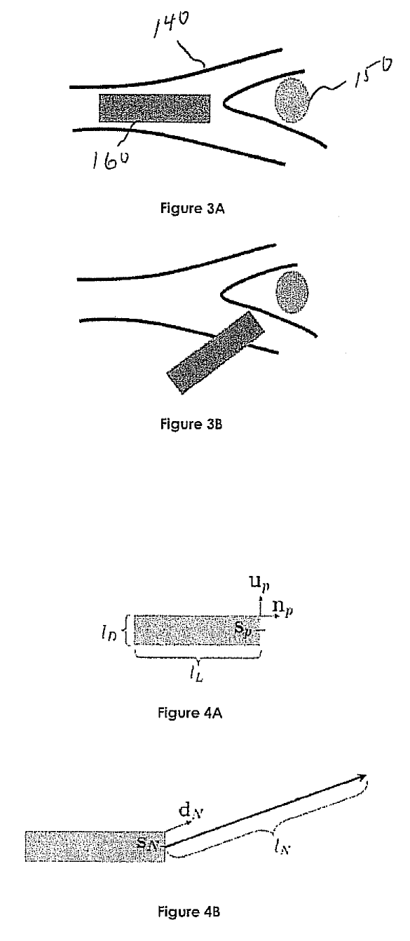

FIGS. 3A and 3B are illustrations of relative geometry of airway tree, ROI, and bronchoscope. The gray bronchoscope tip fits within the airway when oriented toward the ROI at the pose orientation of FIG. 3A. The pose orientation of FIG. 3B, however, is infeasible as the bronchoscope tip protrudes from the airway tree.

The bronchoscopist's ability to sample an ROI is also limited by the physical characteristics of the available bronchoscopic accessories [34]. In general, commercially-available bronchoscopic accessories, such as needles, forceps and brushes have a rigid tip, which collides with the ROI, connected to a flexible body, which allows the accessory to be fed through the bronchoscope. When the physician pushes the rigid accessory tip beyond the bronchoscope, the accessory loses its stiffness, causing placement to become difficult. For this reason, sample orientations must be located relatively close to the ROI. In one embodiment, it is assumed that bronchoscopic accessories have a finite usable length.

In addition to investigating the relationships between the ROI, airway tree and the bronchoscopic devices, planning analysis may be directed to ensure the safety of the patient. For instance, the physician may require that the needle not pierce major blood vessels during the biopsy (FIG. 2B). Because it is difficult to precisely maneuver the bronchoscope to a specific orientation, routes should be chosen that the needle extends within a safety envelope to avoid the obstacles. Likewise, because of the difficulties in aligning the bronchoscope, the physician should be given a large “strike zone” from which to sample the ROI.

The following sections formalizes the pose orientation criteria described above, and describe how to efficiently implement our approach.

Formalizing the Pose Orientation Problem

The task of finding the routes with the best pose orientations may be framed as an optimization problem wherein the objective is to find the pose orientations that maximize the ROI depth of sample subject to physical, device and anatomical constraints. Assuming that the bronchoscope can fit through the airway tree to reach a particular destination, which can be determined using the quantitative analysis methods of Gibbs [6], the relative geometry between the bronchoscope, airway tree, ROI, and obstacles at the route's pose orientation determine the feasibility and appropriateness of the route. Recall from 2.1, the configuration of the bronchoscope tip at the route destination, the pose orientation p, is parameterized by

p={sₚ,nₚ,uₚ,rₚ}. (1)

where sₚ is the location of the center of the bronchoscope tip (FIG. 4A). The orthonormal vectors nₚ, uₚ, and rₚ, form a coordinate system oriented along nₚ, the direction in which the tip is pointing.

FIGS. 4A and 4B show a pose orientation coordinate system and needle vector. The pose orientation shown in FIG. 4A gives the alignment of the bronchoscope. A particular needle vector shown in FIG. 4B may not exactly align with the nominal pose orientation direction, due to uncertainty in the needle orientation.

For typical ROIs, there are often many candidate poses at a given airway-tree location, each oriented toward a different section of the target ROI. Some of the potential poses may be infeasible, given the various route-planning constraints (e.g., the needle may pierce a major blood vessel). Assuming a strategy to find safe, viable poses exists, which is described herein, the search for optimal routes can be restricted to constraint-satisfying feasible poses.

From the subset of feasible poses, those that maximize ROI depths of sample while accounting for the difficulty in precisely aligning the bronchoscope to a particular configuration are sought. Uncertainty in bronchoscopic direction is treated independently from uncertainty in bronchoscopic location. In this embodiment, first assume that the physician can reach a precise position in space, but there exists uncertainty in the direction in which the bronchoscope is pointing, i.e., at the known location sₚ, the needle may deviate from the nominal direction nₚ. Positional uncertainty is accounted for by selecting candidate route destinations from high-scoring, alignment-robust pose neighborhoods.

While the analysis holds for a wide variety of bronchoscopic accessories, now assume the bronchoscopic accessory in use is a needle N. The trajectory of N is modeled by

N={s_{N},d_{N},L_{N}}, (2)

where s_{N}=s_{P} is the origin of the needle at the pose location, d_{N} is the needle direction, and L_{N} is the needle length. A needle, therefore, is simply a vector located at s_{N} pointing along d_{N} with length L_{N} (FIG. 4B). Under our current assumptions, s_{N} known precisely—it is identical to a pose orientation location sₚ, but there is some ambiguity in d_{N} For convenience, d_{N} is calculated using a spherical coordinate system aligned along the pose coordinate system nₚ, rₚ, and uₚ. A particular needle direction associated with pose orientation p is given by

d_{N}(θ,φ)=nₚ cos(φ)+uₚ cos(θ)sin(φ)+rₚ sin(θ)sin(φ), (3)

where θ and φ are random variables. This needle-alignment parametrization represents the probabilistic uncertainty in a natural way. Because physicians may approach the route destination in a variety of configurations, no prior knowledge of the rotation of the bronchoscope at the pose is assumed. In this way, θ is uniformly distributed. While it may be impossible to assume an exact bronchoscope orientation, the physician will generally “try their best” to align the bronchoscope along the pose normal direction nₚ. However, this may not be possible, so the probability of a particular needle configuration decays radially about nₚ, where φ is the angle between the needle direction and nₚ. These assumptions are captured in the probability density function

$$p ( θ , φ ) = \frac{{φ}_{FOV} - φ}{{πφ}_{FOV}^{2}} , \qquad ( 4 )$$

where θε[0,2π] and φε[0,φ_{FOV}]. This function achieves a maximum when the needle is exactly aligned with nₚ and decays linearly over a sampling field of view φ_{FOV}. For a given needle, the function D(s_{N},d_{N},L_{N}) gives the depth of sample,

D(s_{N},d_{N},L_{N})=∫₀^{L}^{_{N}}χ(s_{N}+ld_{N})dl (5)

where χ(m) is an indicator function, χ→{0,1}, stating whether the target ROI exists at location m. The expected depth of sample E_{θ,φ}[D(s_{N},d_{N},L_{N})] over all possible needle vectors at a given pose is therefore

E_{θ,φ}[D(s_{N},d_{N}(θ,φ),L_{N})]=∫₀^{φ}^{_{FOV}}∫₀^{2π}D(s_{N},d_{N}(θ,φ),L_{N})p(θ,φ)dθdφ (6)

where the dependence of d_{N} upon the random variables θ and φ has been again made explicit. Recall that d_{N} is also dependent upon the orientation of the pose directions nₚ, uₚ, and rₚ. E_{θ,φ}[D(s_{N},d_{N},L_{N})] takes a value in the range (0,L_{N}), with larger values reflecting greater expected depths of sample. It is generally desirable to sample as much of the ROI as possible and to obtain poses that maximize Eq. (6).

Until this point, the location of the bronchoscope tip s_{N} has been assumed to be known and exact. To account for location ambiguity, portions of the airway tree with many feasible poses are sought, each of which has a large expected depth of sample over potential needle vectors. This requirement is satisfied by optimizing over “neighborhoods,” or sets of nearby poses, seeking the neighborhood where the expected depth of sample of the M^{th} percentile pose achieves a maximum value.

The optimization problem presented in this subsection provides the framework for determining the best locations in the airway tree from which to sample the ROI. The following section details how the optimization problem can be tractably implemented to find the best candidate routes in a clinically-appropriate timeframe on a standard desktop computer.

Efficient Pose Orientation Implementation

To implement the pose-orientation optimization, two steps are performed in this embodiment. First, the set of candidate pose orientations from which a search will be conducted is determined. For this step, the locations of poses separately from pose directions are found. The pose locations are determined from a sub-sampled set of voxels in the airway-tree segmentation I_{S}, while the directions are chosen so that pose orientations face toward separate “chunks” of the ROT. Once we have determined the set of candidate poses, we examine the set in an efficient manner to find the poses with the largest expected depth of sample. To save computational resources, the search proceeds so that poses with the best chance of having a large expected depth of sample are examined before poses with a lower likelihood of having a large expected depth of sample. However, while poses can be examined in order of potential expected depths of sample, this search strategy still finds the globally-optimal set of pose orientations subject to the assumptions discussed herein.

To determine whether a candidate pose should be included in the search, its viability and safety are examined in a series of efficient feasibility tests. Then, the pose/needle combinations are examined in an efficient order to determine the poses with optimal expected depths of sample. This process is described in detail below.

Candidate Poses

In this embodiment, candidate poses are determined in two independent stages. First, the pose locations are found. Second, a set of representative “chunks” of the ROI target determine the pose directions. The nominal pose direction at a given location is oriented such that nₚ faces toward the one of the ROI “chunks” center of mass.

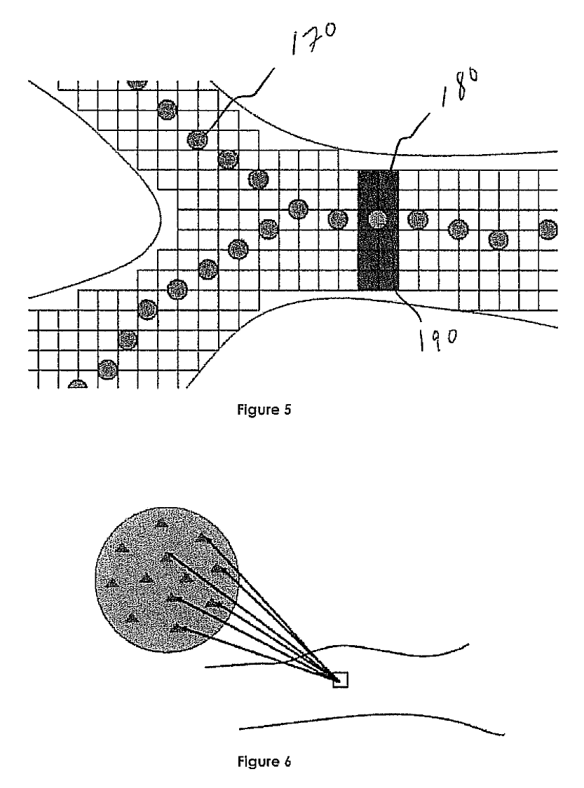

The pose locations correspond to voxel locations in the airway-tree segmentation I_{S}. Here, the conservative nature of I_{S} is beneficial—a feasible pose orientation must be located slightly away from the airway wall so the bronchoscope can fit within the organ. Because pose locations correspond to a route destination in the neighborhood of a particular view site, each voxel in I_{S} is associated with the viewing site it is nearest (FIG. 5). The seemingly simple task of associating voxels with view sites exemplifies how implementation details dictate the clinical feasibility of route planning. By using an octree, the view site/voxel associations are found in under five seconds on a computer that requires over three minutes for brute-force calculations [27].

To save computational resources, not every voxel is considered as a candidate pose location. Instead, the set of voxels associated with a view site is subsampled in raster-scan order so that each view site is associated with at most N_{MAX} pose locations. In one preferred embodiment, N_{MAX}=200, which has been used for all the computations in this document, yields preferred results. However, the invention is not so limited, N_{MAX} may range from 1 to as many sites as can be discretely represented in computer memory_and more preferably from 50 to 400.

FIG. 5 is an illustration of view site/voxel associated pose locations. The smooth outer curves represent the airway surfaces, the interior grid is the airway-tree segmentation, and the circles represent view sites. The shaded voxels 180 are nearest, and therefore associated with, the view site 190. The centers of the shaded voxels 180 are candidate pose locations associated with the view site 190.

At each pose location there are multiple poses, differentiated by the nominal pose direction, with each pose oriented toward a different ROT region. By sampling a sufficiently large number of orientations, expected-depth-of-sample calculations can reasonably cover the ROI. For an ROT broken into a set of K sections, the nominal pose directions nₚ^{i,j}, j=1, . . . , |K| at a given pose location sₚⁱ are unit vectors pointing from sₚⁱ toward the centers-of-mass of subsections of the ROT in K (FIG. 6). The up and right pose directions uₚ and rₚ are arbitrary, so long as they form an appropriate orthonormal system relative to nₚ.

To arrive at the points in K, we partition the ROT voxels into subvolumes by the k-means algorithm [12], with each point in K corresponding to one of the final k-means locations. The number of ROT target locations required to appropriately sample the ROT depends upon the shape of the ROT. For instance, a long cylindrical ROI should have a larger number of targets |K| than a sphere of equal volume, as more of the sphere is visible within a fixed field of view. |K| is therefore proportional to the surface area of the ROT. Finally, requiring |K|≦25 helps ensure reasonable run times. However, the invention is not so limited and other values for K may be used.

FIG. 6 shows an illustration of ROT partitioning via k-means. The triangles show the k-means locations and the square is a voxel in I_{S}, corresponding to pose location. The arrows represent different pose orientations nₚ^{i,j}, j=1, . . . , |K| at a pose location sₚⁱ.

Pose Feasibility

The candidate poses found above may represent unsafe or physically-impractical bronchoscope configurations. A series of tests, applied in order of computational complexity, remove the impractical poses. The following summarizes tests, in order of application. However, in other embodiments of the invention, one or more of the tests may be omitted, and the order may vary.

1. Global Fit—the bronchoscope must be able to reach the route destination associated with the pose orientation.

2. Direction—the pose must be oriented in a physically appropriate manner.

3. Local Fit—the tip of the bronchoscope must remain within the airway.

4. Obstacle—there can be no obstacles within a “no fly” safety region around the pose.

The global fit test determines whether the bronchoscope can reach a given pose orientation by determining if the airways are large enough along the route. This test, performed at the view-site level, is nearly instantaneous.

The direction test helps ensure the pose is oriented in a realizable manner. For instance, a pose in the trachea that is oriented toward the proximal end of the airway cannot be reached—the physician would have to double back to reach this configuration. To satisfy the direction test, the angle between nominal pose direction nₚ and the normal view site direction n must be less than a threshold ψ,

nₚ^{T}n≧cos(ψ). (7)

A straightforward implementation of this test is fast enough for a route-planning scheme. In a preferable embodiment, ψ=65°. This value is used for the results presented in this disclosure.

An efficient implementation is preferred for the local bronchoscopic-fit test. The local-fit test determines whether the rigid bronchoscope tip with diameter l_{D} and length l_{L} (FIG. 4) remains within the airway-tree. The airway-surface model is preferably used to determine these quantities, as it is more accurate than I_{S}. The bronchoscope tip may be modeled as a collection of points C on the cylinder defined by sₚ, nₚ, l_{D} and l_{L} (FIG. 4A) [18, 19]. Directly using the airway-tree surface triangle set to determine whether a point cεC is inside or outside the airway, as is typically done in similar problems, is unwieldy [18, 19, 4]. This calculation requires comparing the position of the test point with the position and inward-facing normal vector of airway-tree surface triangles in an appropriate local neighborhood. Defining the appropriate local neighborhood of airway-tree surface triangles is difficult, however, due to the complicated structure of the continuously bifurcating tree.

Rather than using the airway-tree polygonal surfaces to perform bronchoscope/airway-tree collision, an airway-tree surface likelihood image I_{L}, generated by the method of Gibbs et al. [8, 7] may be used. The airway-tree surfaces lie along the O-grayscale interpolated isosurface of I_{L}. That is, points within the airway tree have negative grayscale values while those outside the airway tree have positive grayscale values. The bronchoscope fit test therefore requires that the trilinearly-interpolated grayscale value in I_{L} of every point contained in C be ≦0. The points in C are spaced no further apart than min(Δx, Δy, Δz) to ensure a proper coverage of the bronchoscope cylinder.

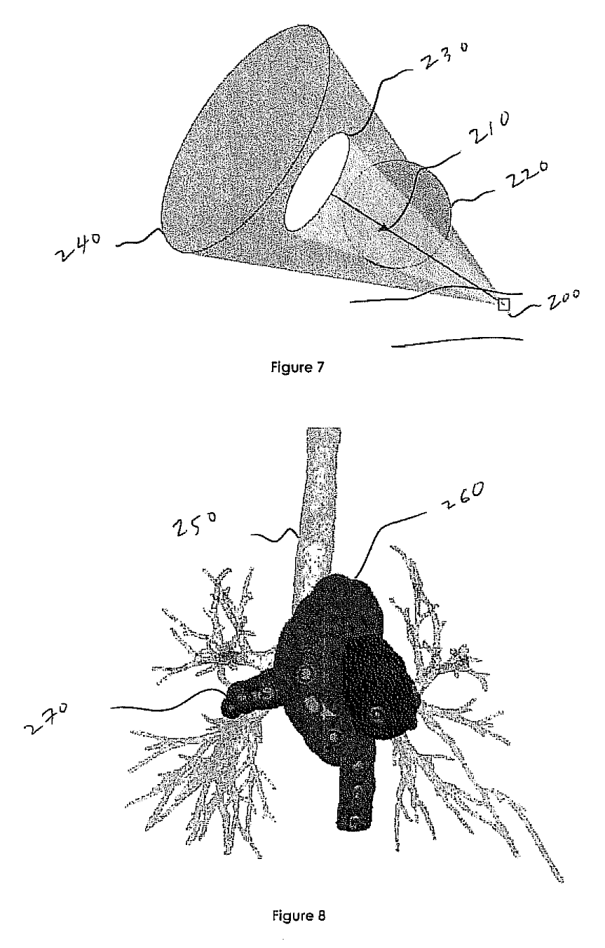

FIG. 7 illustrates the expected depth of sample envelope and safety envelope for a pose. A pose at a segmentation voxel (200) is oriented toward a target section (210) of the green ROI 220. The small cone 230 is the envelope considered for the expected depth of sample calculations. The larger or second cone 240 is the safety envelope. The expected depth of sample envelope is completely contained within the safety envelope.

The final feasibility test, obstacle detection, is typically the most time-consuming. This determines whether there are any obstacles, usually blood vessels, in a “no-fly” region about the pose (FIG. 3). The “no fly” region is similar in shape to the region examined in the expected depth of sample calculations, consisting of a safety envelope of length L_{S} and safety field of view φ_{S} oriented at sₚ along direction nₚ. Because of the risks associated with hitting these sensitive regions, obstacle-avoidance calculations must be exact. Casting a few rays through a segmented obstacle image to check for the obstacle is insufficient. Instead, every voxel within the safety envelope is preferably checked, requiring a significant number of obstacle-image lookups. Furthermore, because the safety envelope is not an axis-aligned rectangular prism, additional overhead is required to determine the voxels to check. Performing the many calculations required to merely determine if a pose is safe is too expensive for clinically-tractable route planning.

Instead of checking voxel locations, polygonal representations of the obstacle surfaces are rendered. To extract the polygonal surfaces, the binary image I_{O} of the obstacle voxels, with grayscale values of 200 and the background having a 0 grayscale value, undergoes a morphological closing with a 6-connected structuring element. The closed obstacle image is then filtered with separable Gaussian kernels, each with a standard deviation ≈0.5 mm. These two steps slightly expand and smooth the obstacle ROI. From the closed, filtered image we extract polygonal obstacle surfaces at the 100 grayscale isosurface via Marching Cubes [20].

The obstacles such as the aorta or pulmonary artery can be decimated by a factor of up to 90% and still retain an appropriate shape. Decimation may be carried out as is known to those of skill in the art and as, for example, as described in the Visualization Toolkit [29].

The obstacles are rendered to a viewing plane aligned along the pose coordinate system, i.e. normal to nₚ. The viewing frustum for the obstacle renderings reflects the required length L_{S} and field of view φ_{S} for the safety envelope. If any portion of the obstacle is present within the rendered viewing plane, the obstacle is within the safety envelope and the examined pose is therefore unsafe. A software implementation of surface-based obstacle detection is almost as time-consuming as the voxel-based approach. Fortunately, the graphics processing unit (GPU) in standard computers provides a highly-optimized hardware implementation for rendering polygonal triangles within a viewing frustum. A preferred embodiment of the invention uses the OpenGL API to interact with the GPU [35]. Obstacle triangles are assigned “names” and rendered to an off-screen selection buffer at a particular pose orientation. OpenGL then returns a list of obstacles encountered in the viewing frustum. If no triangles are within the viewing frustum the returned list of names is empty, indicating a safe pose orientation.

Because the obstacle rendering time scales with the number of triangles present in the obstacle model, it is preferable to avoid drawing any triangle that can safely be omitted. In most cases, a large number of triangles in the already-decimated obstacle surface can be discarded without affecting the results. For a pose orientation to receive a non-zero expected depth of sample, the pose orientation must be within L_{N} of the ROI surface. For safety's sake, the depth of the viewing frustum L_{S}>L_{N}. The only obstacle triangles that could ever be rendered for a pose with a non-zero expected depth of sample are, therefore, those within L_{S} of the ROI surface. FIG. 8 shows a typical aorta and pulmonary artery segmentation. For this case, the original non-decimated aorta surface consists of 187,756 triangles and the non-decimated pulmonary artery consists of 130,540 triangles. After decimation and removing all triangles ≧L_{S}=22.5 mm from the ROT surface (obscured by the vessels), only 2,032, or ≈0.6% of the original obstacle triangles need be rendered for route planning. Reducing the number of obstacle triangles to be rendered is preferred for clinically-feasible execution times.

FIG. 8 shows a global rendering of the airway tree (250), aorta (260), and pulmonary artery (270). If a candidate pose passes each of the feasibility tests, it may be considered in the expected depth of sample calculations, detailed in the upcoming subsection.

Expected Depth of Sample Computation

With the candidate pose orientations defined and efficient methods to test the feasibility of the candidate orientations, the task now is to calculate the expected depth of sample (6). Because ROIs are not given by functional expressions, the integrals of (6) cannot be directly evaluated. Instead, it is preferable to use discrete approximations of the expected depth of sample integral.

To approximate the expected depth of sample for a pose at location sₚⁱ and nominal directions nₚ^{ij}, uₚ^{ij}, and rₚ^{ij}, rays are cast in M needle directions d_{N}^{ijk}, k=1, . . . , M, at s_{N}ⁱ=sₚⁱ, with each direction parameterized by an angular offset about the nominal pose direction nₚ^{ij}, up direction uₚ^{ij}, and right direction rₚ^{ij} per (3). The angular offsets are contained in vectors

Θ=(θ₁, . . . , θ_{M}) and Φ=(φ₁, . . . , φ_{M})

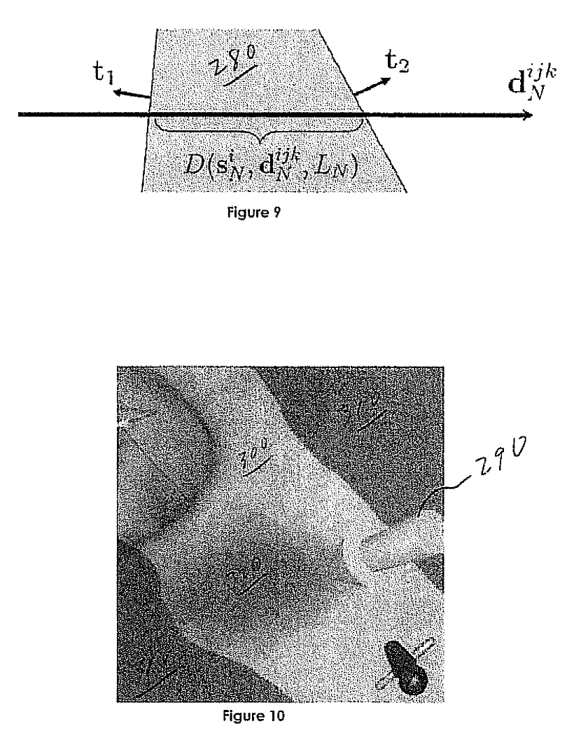

with M the number of distinct needle rays in the approximation. In a preferred embodiment, the depth of sample for a single needle ray is computed from a polygonal surface of the ROI. The intersection of a needle ray with ROI triangles gives locations where the needle is either entering or exiting the ROI (FIG. 9). The inner product of the ray direction with the outward-facing normal t of a surface triangle disambiguates whether the needle is entering or leaving the triangular surface. If t^{T}d_{N}^{ijk}<0, the needle is entering the ROI, otherwise it is exiting. The entering and exiting triangle intersections are examined in order to determine the overall depth of sample for the needle ray. Not all triangles need be examined for intersection with a particular ray in the expected depth of sample calculations. Similar to obstacle detection, the GPU provides specialized hardware acceleration for this task. Using the same selection-mode rendering as before, this time with a FOV of only 5°, the triangle intersection calculations performed in software can be dramatically reduced when compared to examining all triangles on the ROI surface. Another embodiment for calculating a needle's depth of sample is performed in the CPU by lookups at discrete locations along each needle. Choosing one embodiment for calculating the depth of sample, in the GPU or CPU over the other is dependent upon the performance of a particular GPU or CPU in a specific computer and is selected in terms of which performs the calculations faster.

To approximate the overall integral, each individual depth-of-sample D(s_{N}ⁱ,d_{N}^{ijk},L_{N}) is weighted by ωₖ, to reflect the pdf of random variables θ and φ, with Σₖ₌₁^{M}ωₖ=1. The weighted average of the depths-of-sample of individual rays yields an approximation to the expected depth of sample:

$${E}_{θ , φ} [ D ( {s}_{N}^{i} , {d}_{N}^{ij} ( θ , φ ) , {L}_{N} ) ] \approx {\sum }_{k = 1}^{M} {ω}_{k} \cdot D ( {s}_{N}^{i} , {d}_{N}^{ijk} , {L}_{N} ) . \qquad ( 8 )$$

FIG. 9 show the depth of sample D(s_{N}ⁱ,d_{N}^{ijk},L_{N}) of a single needle. The needle is oriented along direction d_{N}^{ijk} and the outward-facing normals of the two intersected triangles are t₁ and t₂. The shaded area 280 indicates the interior of the ROI volume.

Instead of computing all needle rays, in order, per (5), it is preferred to examine a group of potential poses orientations Sₗ={p₁, . . . , pV}, associated with a view site, simultaneously. Prior to consideration in the expected-depth-of-sample calculations, all poses in Sₗ have passed the feasibility tests.

In this embodiment, because only the F best pose orientations in the top M^{th} percentile of pose locations associated with a route destination are sought, the following are considered, in order, the needles with the largest weights at the poses with the largest potential expected depth of sample. For example, prior to examining the poses in Sₗ, the largest achievable expected depth of sample for a particular pose¹ is uncertain.

After examining a single needle ray at each pose in Sₗ, however, it is preferable to hone in on the poses that achieve the best expected depth of sample. For example, the needles at some poses may achieve maximal depth of sample, while others may have zero depth of sample. Once the poses with the largest depth of sample have been determined, and therefore the poses with the highest-potential expected depths of sample have been determined, more needle rays at these most promising poses are examined. By only examining needles associated with poses that posses the largest expected depth of sample upper bounds, many calculations are expected to be saved as compared to a brute-force approach. Furthermore, if enough route destinations with large expected-depth-of-sample pose orientations (≧1) have already been found in accordance with this embodiment of the invention, the minimal score to care about may be identified or bound. In this way, no time is wasted examining poses where the achievable expected depth of sample <lₘᵢₙ. ¹The highest achievable expected depth of sample can be bounded from above based on the distance from the pose to the surface of the ROI (Section 2.2.6).

Algorithm 2.1, shown below, provides detail for the expected-depth-of-sample-calculation strategy of poses associated with a particular route-destination view site. The inputs to the algorithm are Sₗ, the set of feasible poses at a view site; F, the number of different pose locations required in the M^{th}-percentile expected-depth-of-sample calculations; lₘᵢₙ, the smallest expected depth of sample of interest; Θ and Φ, a list the discrete needle direction offsets; ω the weights associated with each needle offset; and L_{N}, the length of the needle. The output of the algorithm, S_{F}, is a set of poses, |S_{F}|≦F each at a different location, (if sₚⁱ,sₚ^{j}εSₚ and i≠j, then sₚⁱ≠sₚ^{j}). If there are <F poses at different pose locations that achieve expected depths of sample ≧lₘᵢₙ then the algorithm returns no poses (S_{F}=Ø).

In addition to use of a needle to obtain a tissue sample, another embodiment of the present invention includes use of other instruments or appliances such as, for example, a coring needle, brush, forceps, RF ablator, cryo-ablator, oxygen sensor, electrical sensor, implant delivery catheter, aspiration device, fluid delivery device, or temperature sensor. Characteristics (e.g., dimensions, functional attributes, etc.) of such appliances may be used as input in the present invention to optimize associated applications and poses. Additionally, in one embodiment of the present invention, the make and model number of the appliance is accepted by the workstation. The workstation is adapted to retrieve device characteristics and feature information corresponding to the model in order to determine candidate routes, and poses that best serve the appliance, or that meet user identified criteria.

Additionally, in some cases, the invention may indicate that no candidate poses or routes exist given the appliance, obstacles, endoscope, and or other criteria. This may be visually indicated to the physician.

The next subsection describes the manner in which candidate route destinations are examined to efficiently find the routes whose pose orientations safely maximize the expected depths of sample.

Algorithm 2.1 S_{F} = FindBestPosesInSet(S_{I}, F, lₘᵢₙ, Θ, Φ, ω, L_{N})

for i ← 1, number of pose locations in S_{I} do

for j ← 1, number of feasible pose directions at pose location sₚⁱ do

l_{B}[i][j] ← L_{N} {keeps track of upper-bound expected depth of sample (EDOS) at pose
{sₚⁱ,nₚ^{ij},uₚ^{ij},rₚ^{ij}} ∈ S_{I} }
f[i][j] ← 0 {keeps track of last needle direction examined at pose {sₚⁱ,nₚ^{ij},uₚ^{ij},rₚ^{ij}} ∈ S_{I} }
push pose {sₚⁱ,nₚ^{ij},uₚ^{ij},rₚ^{ij}} onto Q {Priority queue Q returns pose orientations in decreeing
order of l_{B}[i][j]}

end for

end for
while |Q| ≧ 0 and |S_{F}| < F do

{sₚⁱ,nₚ^{ij},uₚ^{ij},rₚ^{ij}} ← top(Q)
pop(Q)
if a pose at position sₚⁱ ∉ S_{F} then

if ƒ[i][j] = |Θ| (all potential directions have been examined) then

insert pose {sₚⁱ,nₚ^{ij},uₚ^{ij},rₚ^{ij}} into S_{F} {this is one of the top poses}

else

k ← f[i][j] + 1 {Examine the next direction at this pose}
f[i][j] ← k
Compute d_{N}^{ijk} per 3 for coord. sys. of pose {sₚⁱ,nₚ^{ij},uₚ^{ij},rₚ^{ij}} and needle offsets
Θ[k], andΦ[k]
Compute D(s_{N}i,d_{N}^{ijk},L_{N}) per 5 at S_{N}ⁱ = sₚⁱ
Y ← w[k]·D(s_{N}i,d_{N}^{ijk},L_{N}) {Weight the pose's DOS}
l_{B}[i][j] ← l_{B}[i][j]−(L_{N}·ω[k]−Y) {Update this pose's highest achievable EDOS.}
if l_{B}[i][j] ≧ lₘᵢₙ then

push {sₚⁱ,nₚ^{ij},uₚ^{ij},rₚ^{ij}} onto Q {Highest achievable EDOS must be large enough}

end if

end if

and if

end while
if |S_{F}| < F then

S_{F} ← {Only return a set of poses if enough good ones were found}

end if
return S_{F}

Route Destinations with Best Pose Orientations

With an efficient way to examine pose orientation feasibility and find the M^{th} percentile pose orientations associated with a candidate route destination, the final task is to determine an efficient order in which to examine the candidate route destinations. The expected depths of sample of poses at a distance ≧L_{N} mm from the ROI surface is zero, so route destinations with no pose orientations ≦L_{N} mm from the ROI need not be examined. Likewise, the orientations nearest the ROI have the largest potential expected depths of sample. Of course, the ROI, airway tree, and bronchoscope geometry determine the feasibility and realizable expected depths of sample of a particular pose, but the distance of a pose to the ROI surface gives an upper bound on the expected depth of sample of the poses at a route destination with minimal computational complexity. In this embodiment, route destinations are ordered in a list b by the distance between the ROI surface and the nearest pose location associated with a route destination, given by l(b[i]). As such, if i≦j, then l(b[i])≦l(b[j]). To determine this ordering, an octree data structure is utilized.

The route destination view sites are examined in the order of the indices in b. The calculations to the order in which route destinations are considered are similar to those previously disclosed, when the route destinations nearest the ROI were found [13]. An important difference is that the distances are calculated between the pose orientations associated with the route destination and the ROI rather than the distance between the route destination itself and the ROI.

In this embodiment the top N optimal routes (or, for example, first routes), such that no pair from among the N best routes are within a “neighborhood” of one another. The neighborhood can be defined such that routes terminate on different branches, paths, or the cumulative distance along the medial axes between view sites is > some threshold. A candidate route must, therefore, have associated poses such that the M^{th}-percentile expected depth of sample is ≧ than all of its neighbors. The M^{th}-percentile expected depth of sample of the candidate route destination must also be > the current N best route destinations. As soon as either of these conditions cannot be met, FindBestPosesInSet can terminate early—the route under examination cannot be one of the N optimal. Algorithm 2.2 provides more detail for the search strategy.

Algorithm 2.2 R_{best} = FindBestRouteDests(S, b, N, M, Θ, Φ, ω, Z_{N})

for i ← 1,|b| do

j ← b[i] {j is the route destination index at b[i]}
e[j] ← 0 [e|j] is the EDOS of the M^{th} percentile pose at the current route destination index.}

end for
R ← {R is the set of the N best poses}
rₘᵢₙ ← 0 {rₘᵢₙ is the smallest EDOS ∈ R}
for i ← 1,|b| do

e_{neigh} ← 0 {Variable to keep track of the maximum EDOS in the neighborhood of the current
route destination}
for all j ∈ Neigh(b[i]) do

e_{neigh} ← max(e_{neigh},e[j]) {Find the highest EDOS in the neighborhood}

end for
lₘᵢₙ ← max(e_{neigh}, rₘᵢₙ) {The EDOS of this route dest. must be better than everyone in
neighborhood and the worst; EDOS of top destinations already found}
F ← number of pose locations to find, corresponding to pose locations in the M^{th} percentile
and above for b[i]
O_{All} ← all pose orientations in S associated with view site b[i]
O_{Feas} ← pose orientations ∈ O_{All} that pass all feasibility tests of subsection 2.2.4
O_{Best} ← FindBestPosesInSet(O_{Feas},F,lₘᵢₙ,Θ,Φ,ω,L_{N}) {Got the best poses around view
site b[i]}
if O_{Best} ≠ and minimum EDOS pose ∈ O_{Best} > rₘᵢₙ then

insert minimum EDOS pose ∈ O_{Best} into R {maximum sine of R is N, so if |R| ≧ N, the
pose with the smallest score gets removed from R}
e[i] ← minimum EDOS pose ∈ O_{Best}
if |R| ≧ N then

rₘᵢₙ ← minimum EDOS of pose ∈ R

end if

end if

end for
R_{best} ← R
return R_{best}

The input to Algorithm 2.2, FindBestRouteDests, is the set of all potential pose orientations S with each pose orientation εS associated with a route destination; b is a list of candidate route destinations ordered in increasing distance to the ROI; N is the number of route destination/pose orientation combinations to find; Θ and Φ give the discrete angular needle offsets used in the expected-depth-of-sample calculations; ω is a list of the weights for each angular offset; and L_{N} is the usable length of the needle.

The output of Algorithm 2.2, R_{best}, contains up to N route destinations, sorted in order of the M^{th}-percentile expected depth of sample at each view site. There may be <N non-zero expected depth of sample route destinations in R_{best}, depending upon the geometry of the ROI, bronchoscope, airway tree, and obstacles, and the neighborhood definition used in Algorithm 2.2. If no acceptable routes are found, this indicates the procedure may be too risky or the bronchoscope may not be able to maneuver appropriately in the airway tree. The calculations can then be performed with a smaller safety envelope or a larger needle length if the physician feels the potential reward of diagnostically sampling the ROI justifies these changes.

For the pose orientation calculations of this section to be useful, they must be conveyed to the physician. The following subsection describes a visualization system in which the calculated pose orientation, obstacles, and ROI depths of sample are presented in real time.

### Route Destination Visualization

Poses may be presented to the physician variously. In one embodiment of the invention, poses are presented to the physicians by a 4-mm long arrow 290 (FIG. 10). The location and direction of the arrow 290 corresponds to the pose orientation calculated by the methods in the previous section. To safely and effectively sample the ROI, the physician should align the needle of the bronchoscope along this arrow.

FIG. 10 is an illustration of the pose orientation arrow. The arrow 290 shows where the tip of the bronchoscope should be aligned to optimally sample the ROI (case 20349.3.9). The regions 300 depict the virtual airway walls, the regions 310 show locations where obstacles (in this case, the pulmonary artery or aorta) are nearby and the region shows the target ROI 320, which is actually located beyond the airway wall. Each of the regions, ROI, obstacles, arrows, icons, and other markers or anatomical features may be presented visually. Various colors, color intensities, and or symbols or other types of indicators may show or indicate an optimum pose. Indeed, the visual presentation of the information may vary widely. In a preferred embodiment, airway walls are shown in pink, the obstacles are shown in red, the ROI is shown in green, and as the pose is optimized, the intensity of the green ROI and margin is adjusted.

Because the physician may not be able to precisely align the bronchoscope with the suggested pose orientations, or may want to sample lesions from multiple directions during a procedure, additional visual cues convey obstacle locations and ROI depths of sample. Within this visual representation of the anatomy, the physician can freely navigate in the virtual bronchoscopic world, perceiving the depth of sample and possible obstacle locations at any endoluminal pose orientation, not just the predetermined optimal pose. During live guidance, a registration method (e.g., the method described in Merritt et al.) can work in conjunction with the bronchoscopic visualization [24, 23]. With the virtual world aligned with the real world, the physician can evaluate the efficacy and safety of sampling at any achievable pose orientation within the patient's airways.

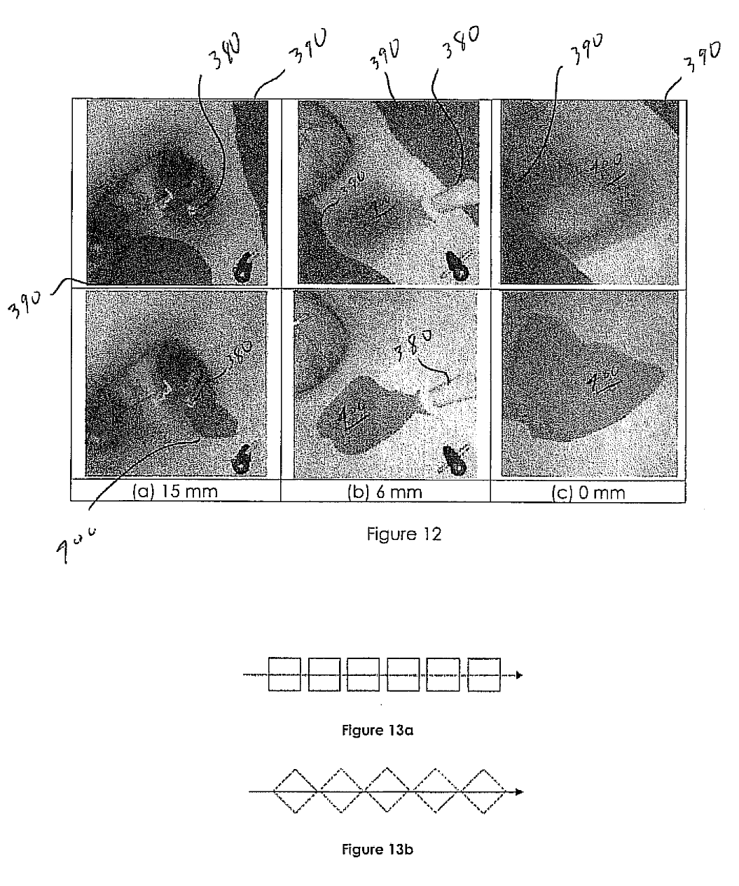

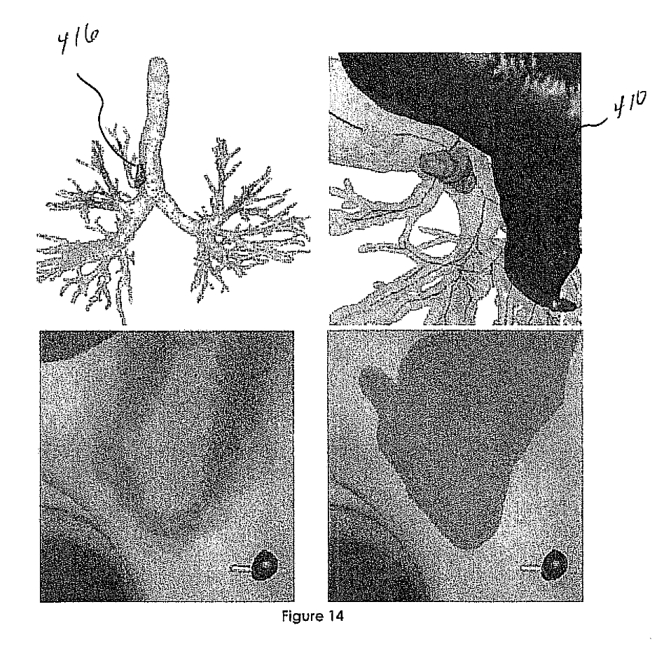

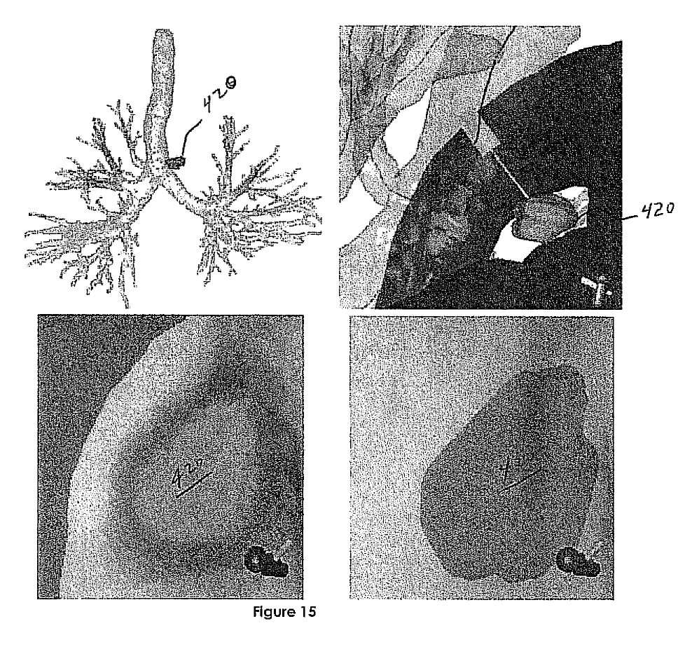

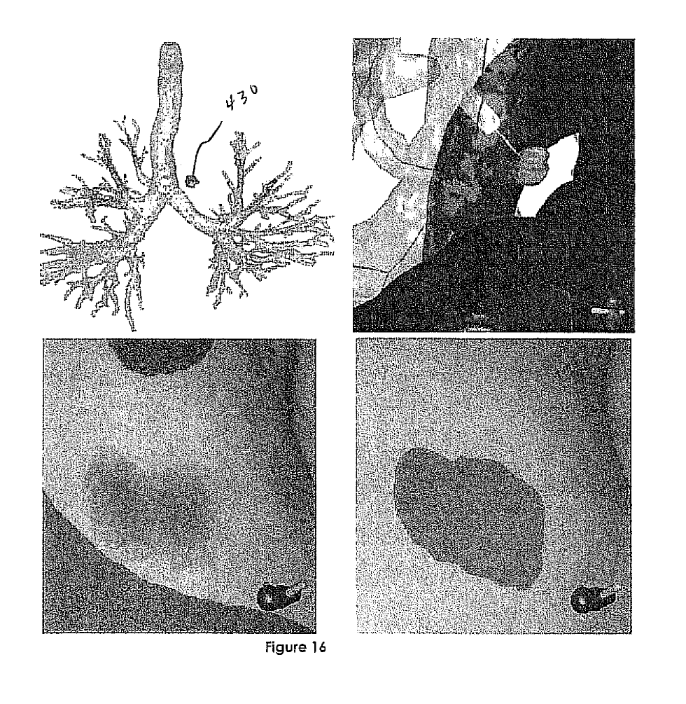

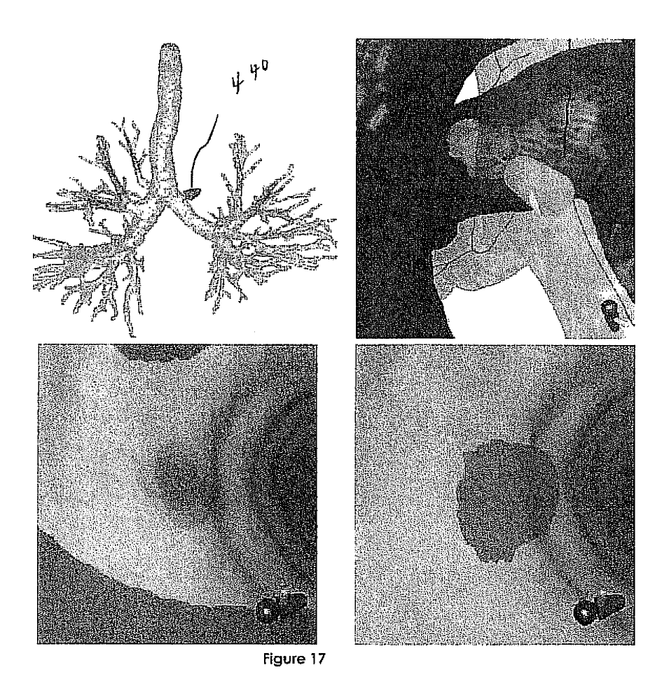

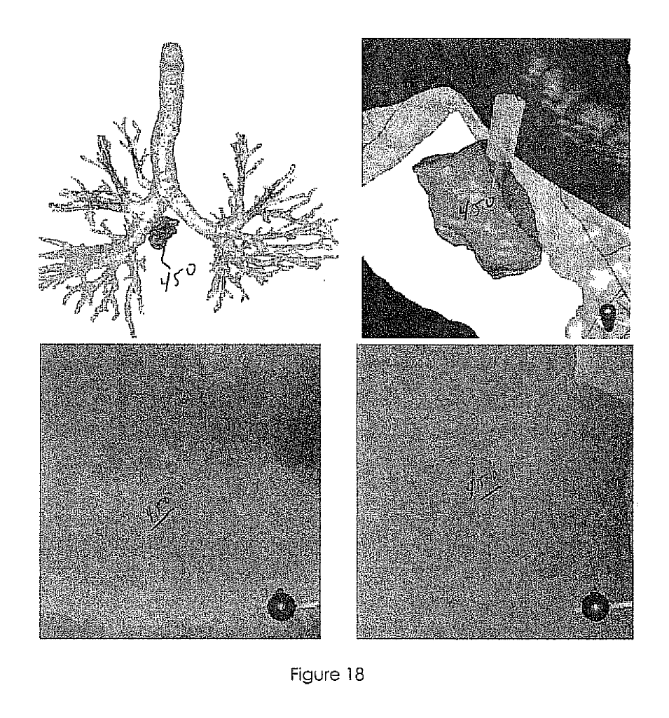

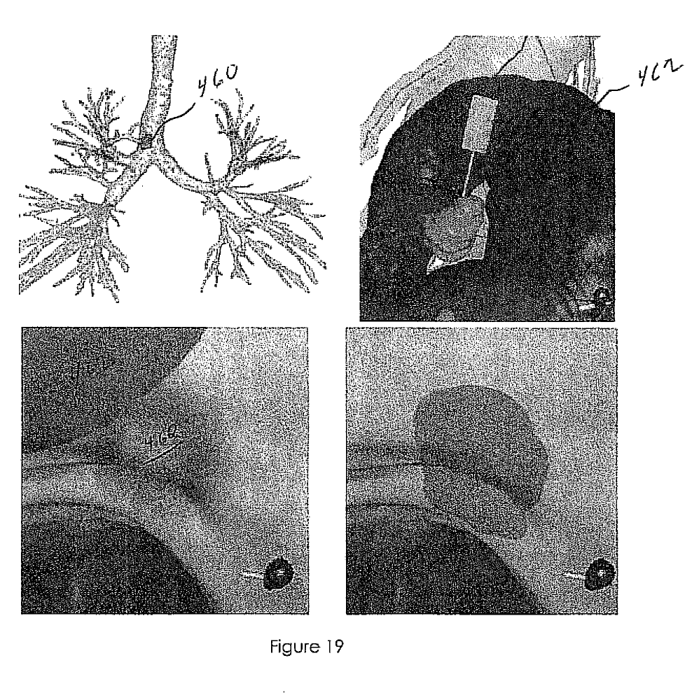

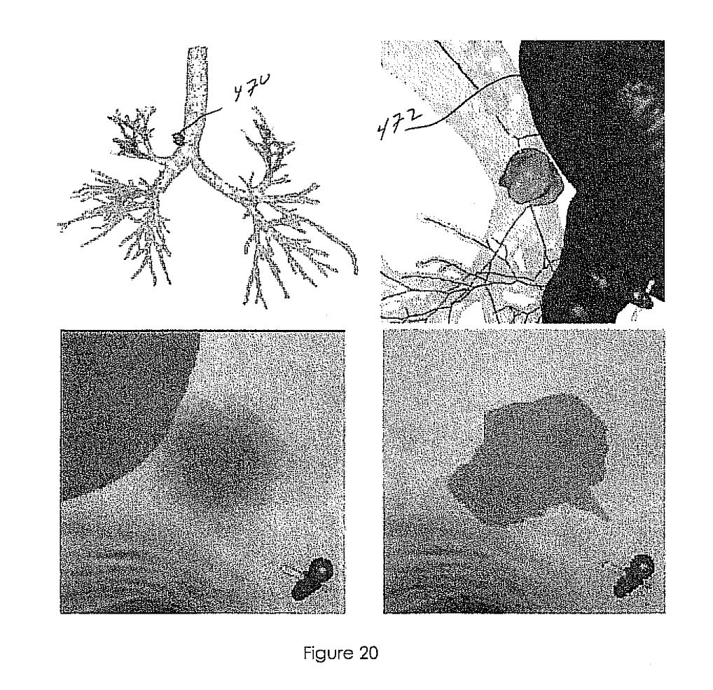

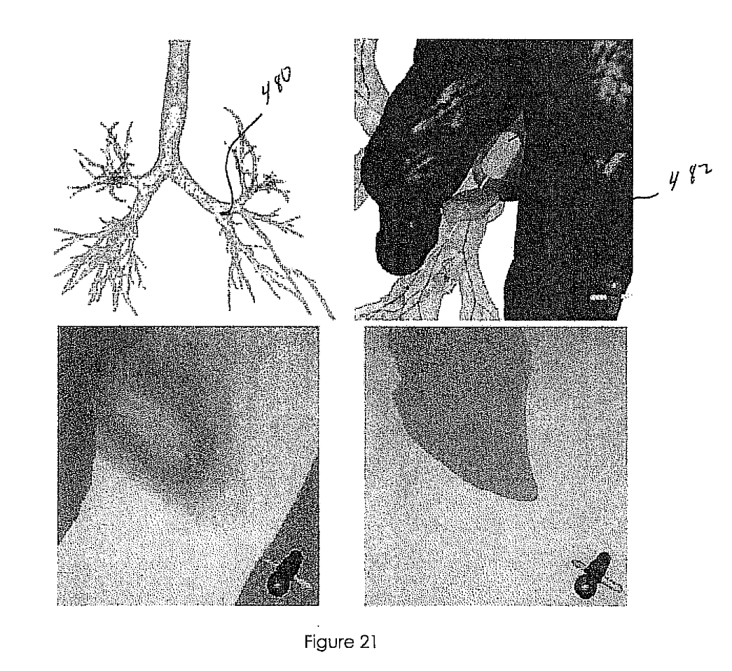

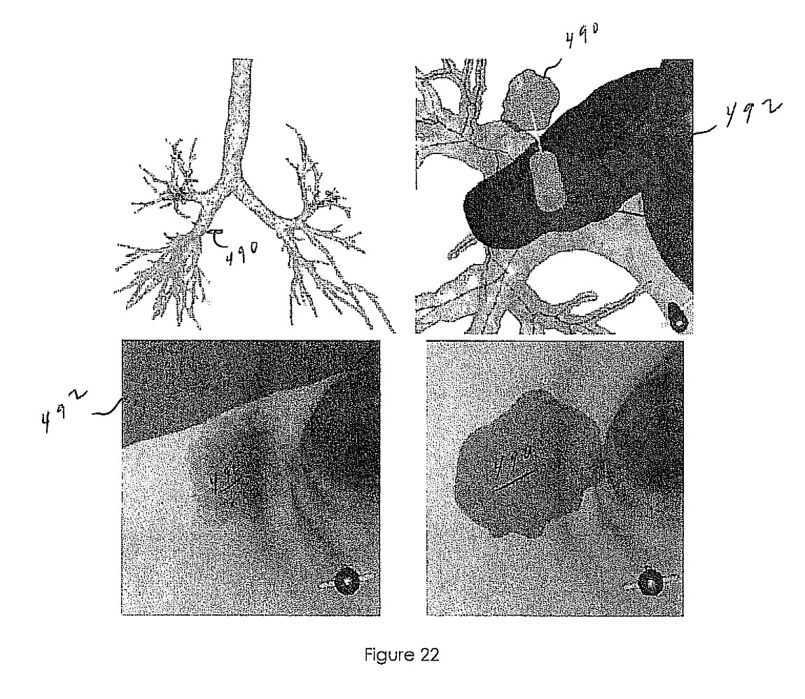

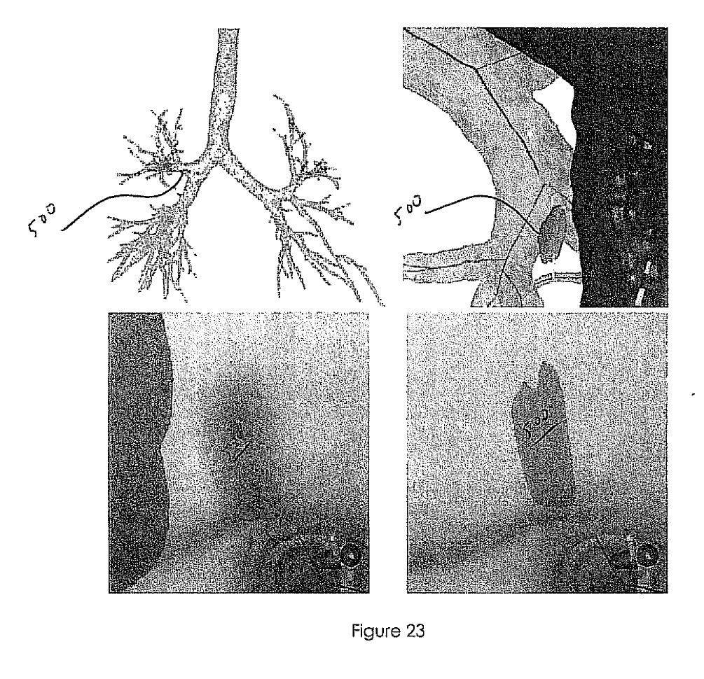

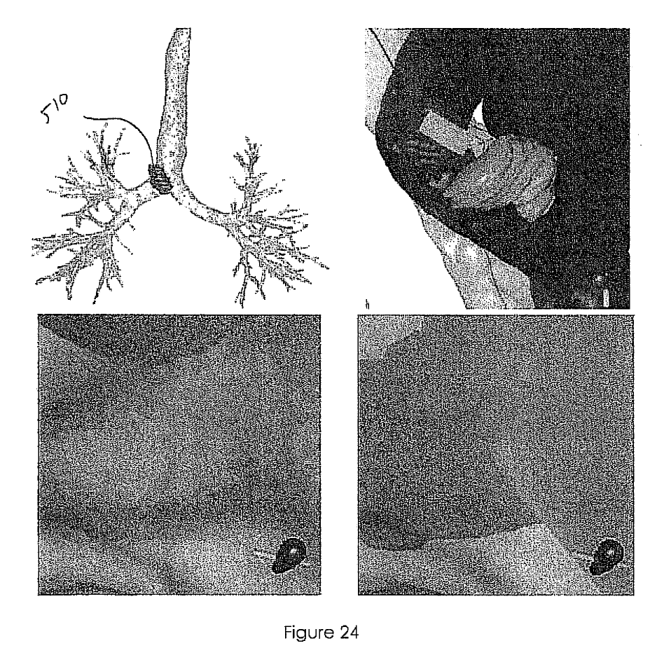

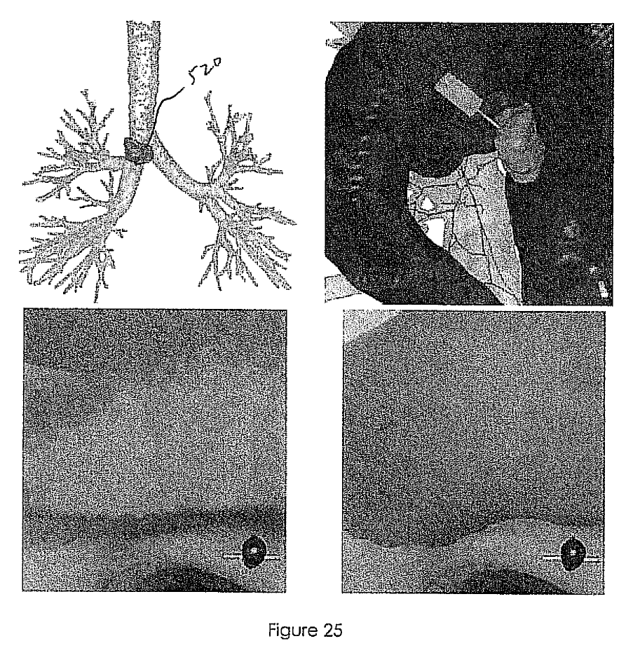

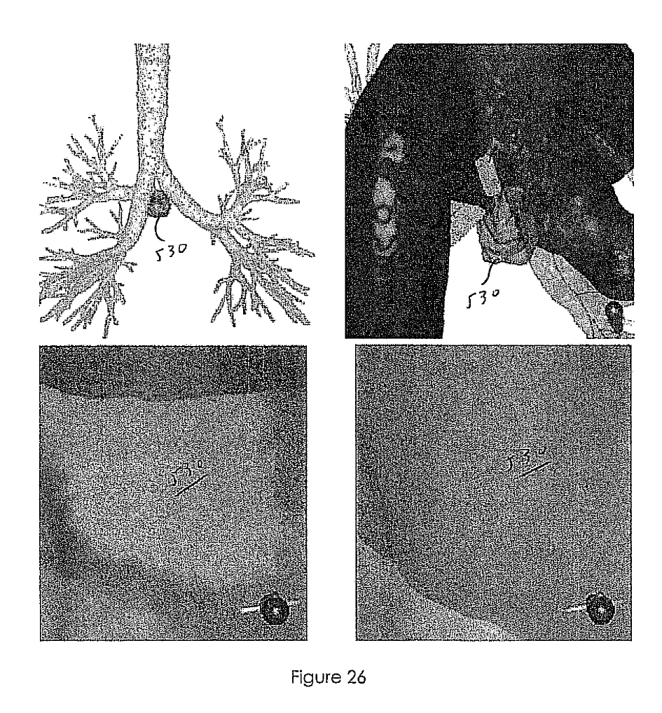

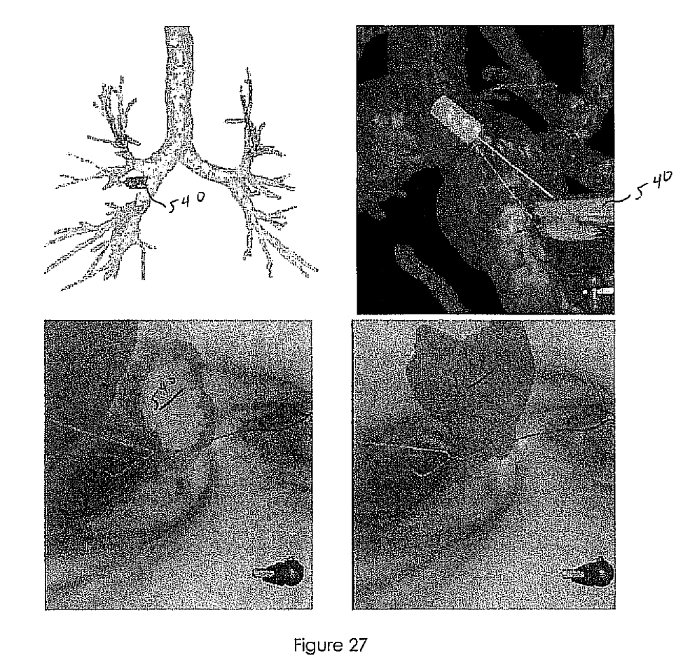

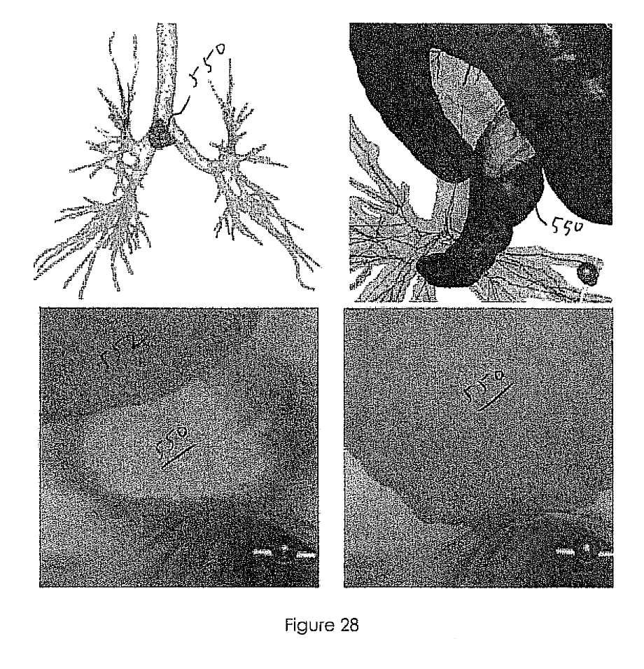

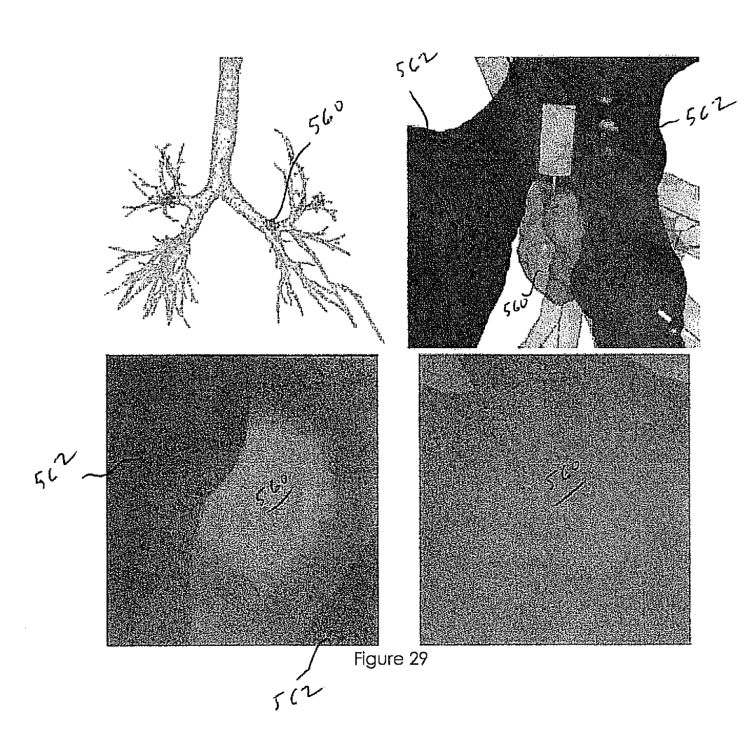

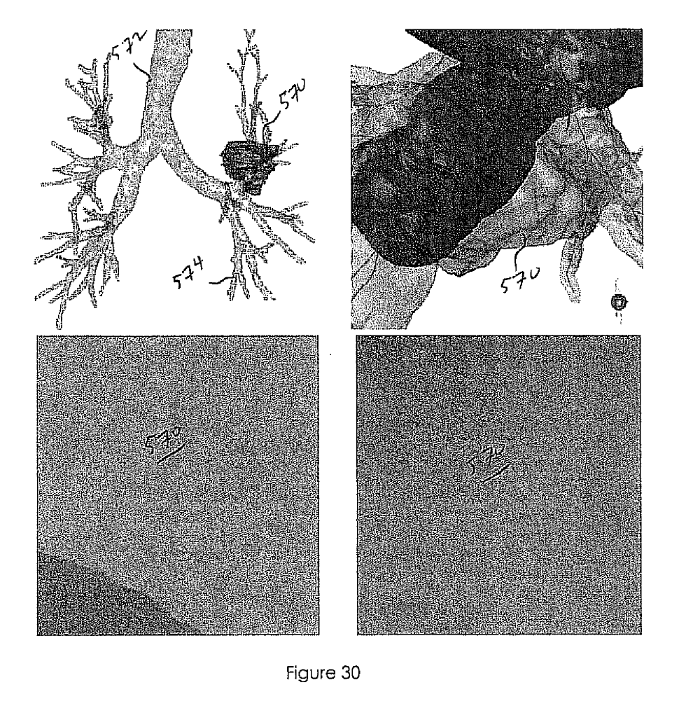

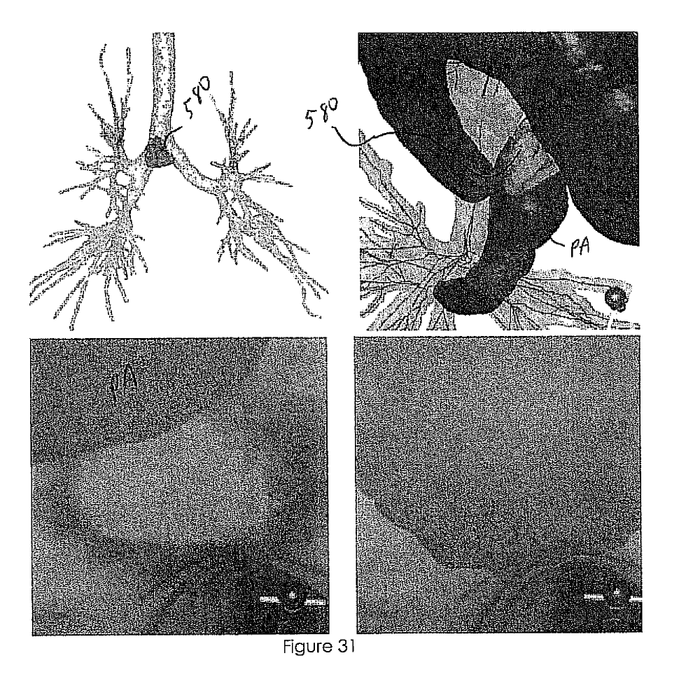

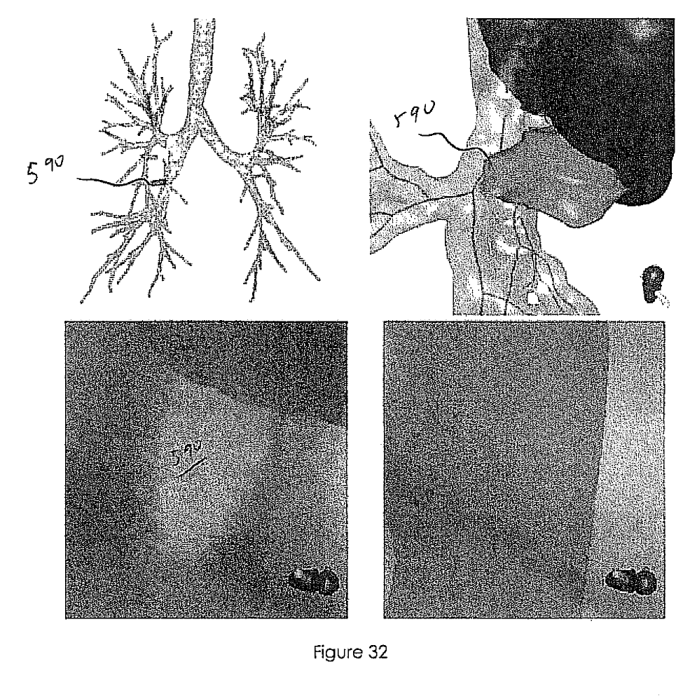

To convey the planning data, the obstacles and ROI depths of sample are represented by colors blended into the VB scene (FIG. 12). Obstacles 390 may appear as intense red structures, and the ROIs 400 may appear as a green target, with the intensity of the green color proportional to the previously-computed best-achievable depth of sample at a given pose. When rendering the VB scene, obstacles takes precedence over ROIs. That is, if an obstacle is visible within a safety distance of L_{S} mm from the virtual bronchoscopic camera location, obstacles are rendered regardless of the presence of the ROI. However, the targets, obstacles, and pose indicators may vary widely and the invention may include a wide variety of geometrical shapes, icons, animations, markers, symbols, colors, color intensities, audible signals, including but not limited to arrows, circles, letters, points, lines, highlights, outlines, etc.

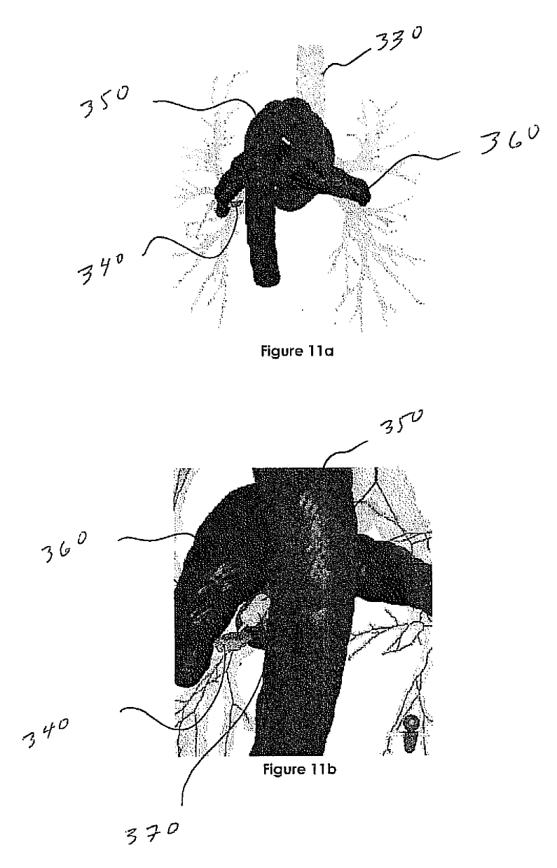

FIG. 11 shows an overview of the case illustrating mediastinal visualization in case 20349.3.9, ROI 2 (FIG. 6). FIG. 11A shows the airway tree (1102), ROI (1104), aorta (1106), and pulmonary artery (1108). This view is from behind the patient so that the ROI is not obscured by the obstacles. The close-up view of FIG. 11B shows these same structures, with the suggested pose orientation calculated using a 4.9 mm bronchoscope with a 10 mm rigid tip.

FIGS. 12A, 12B and 12C show the endoluminal depth of sample and obstacle visual cues for case 20349.3.9, ROI 2 (see FIG. 11). The distances below each view give the remaining distance until the suggested pose orientation, indicated by the 4 mm long arrow. The views on the top of FIGS. 12A, 12B and 12C show the new visualization approach, compared with the previous views on the bottom of FIGS. 12A, 12B and 12C, respectively. In the new views, values in red can indicate the pulmonary artery or aorta (obstacles 390) are within 22.5 mm of the virtual camera. The position in each view shows where the extraluminal obstacles 390 are located. The ROI 400 is indicated in shades of green. The shades of green corresponding to the ROI 400, which is first visible in FIG. 12B. The shades of green get brighter as the physician nears the ROI, indicating the ROI depth of sample at the current pose orientation.

The depth of sample renderings of FIGS. 10 and 12 must be generated quickly so the physician or technician can interact with the VB scene without annoying lags. This suggests the need for hardware acceleration on the GPU. To this end, it is preferable to use additive alpha blending in OpenGL [35]. In additive alpha blending, the GPU accumulates pixel color values in a buffer initially consisting of all zero values. Each object in the scene is then rendered, adding the object's RGB color value, scaled by some constant (alpha) to the existing pixel values. For instance, if existing the RGB value a pixel in the buffer is given by R_{D} and the RGB value of a rendered object at the pixel location is R_{S} and the alpha value is α, then the color of the pixel R_{F} after a single object is

R_{F}=R_{D}+α·R_{S} (9)

The overall rendered image is generated after performing this process for every ROI section, accumulating color values in each pixel in the GPU buffer after rendering each object.

If the ROI is broken into small sections, with each section assigned the same color and alpha values, the net effect of the blending will be bright pixels where many ROI sections are rendered and dark pixels where few ROI sections are rendered. The relative brightness of the blended ROI sections is proportional to the largest-achievable depth of sample, which is what we desire. Care must be taken, however, when performing these calculations. The shape of the representative ROI sections has an effect on the rendered ROI brightness. For instance, if an ROI is represented by voxels, the rendered brightness is dependent upon the orientation from which the ROI is viewed (FIG. 13). Intuitively, the brightness increases one intensity level with each voxel encountered. This figure illustrates that even though the depth of sample is the same for both sets of pixels, the orientation aligned along the voxel coordinate system appears brighter than the orientation that passes through the voxel diagonals as more voxels contribute to the overall pixel brightness in the coordinate-aligned pose. This effect is exacerbated in 3D with anisotropic voxels, as the longest voxel diagonal is typically significantly greater than the minimum voxel dimension.

To address the problems associated with rendering ROI voxels for expected depth of sample visualization, polygonal approximations are preferably rendered as spheres, as these structures have the same perceived depths of sample regardless of the orientation from which they are viewed. To maintain orientation-independent brightness, the spheres are isotropically spaced.

FIGS. 13A and 13B show illustrations of the effect of ROT section geometry on rendered brightness. The arrows represent a ray along which a single pixel's brightness is calculated. The pixel shown in FIG. 13A will appear brighter than in FIG. 13B as the ray of FIG. 13A intersects 6 voxels instead of the 5 of ray FIG. 13B.

The isotropically-spaced spheres are larger than a single voxel for computational tractability. As such, the color and alpha values of each sphere cannot always be identical, as all spheres typically do not represent the same ROI mass. That is, spheres containing more ROI mass should be brighter than those that do not. To determine the mass in sphere Sᵢ, the ROI segmentation is searched and the fraction of the sphere volume fᵢε[0,1] that is filled with ROI voxels is determined. Isotropic spheres placed 1.5 mm apart from one another, on center, yield visually-pleasing depth-of-sample cues and can be rendered without noticeable lag for even large mediastinal ROIs. The spheres overlap to avoid gaps that arise if the spheres were packed as balls in a volume. However, while the above values have been found to be useful, the invention includes additional values and is only limited as recited in the appended claims. Discussed below is an embodiment to determine color and alpha values for each sphere.

OpenGL can only accumulate color values in the buffer at a finite level of precision, which is dependent upon the capabilities of the GPU. Because of this quantization, a small color value, or any color value weighted by a small alpha value, is treated as zero. Such an event results in the color not contributing to the pixel value. For example, if the ROI's RGB value is (120, 25, 10) and color channels saturate at a value of 255, the blue and green channels could easily be quantized to zero when scaled by a small alpha value. To get around this issue, a single color, (e.g., green) for ROIs may be used, while red may be used for the obstacles. In a preferred embodiment, a bright green means “go”. This is a preferred natural cue for the physicians to follow. However, the visual cues and color schemes may be varied and the invention is only limited as recited in the appended claims.

Another challenge addressed in the present invention is to determine to what depth of sample saturates the channel. In other words, how much ROI needs to be along a ray to see the brightest possible green pixel? For large ROIs, this value is merely the needle length L_{N}. However, for smaller ROIs, the ROI would not saturate from any pose and even the best sample orientation would appear dim green and be perceived as a bad location. Accordingly, the fractional weight of each sphere is scaled fᵢ by

$$\frac{1.5}{{l}_{D}} ,$$

where l_{D} is the nominal depth of sample along the pose with the highest expected depth of sample and 1.5 accounts for the spacing of the spheres. In almost all cases, the depth of sample along the optimal pose direction is > than the expected depth of sample of the optimal pose.

The RGB value assigned to a sphere is (0,

$$\sqrt{255 \cdot \frac{1.5 \cdot {f}_{i}}{{l}_{D}}} ,$$

0) and alpha is

$$\sqrt{255 \cdot \frac{1.5 \cdot {f}_{i}}{{l}_{D}}} .$$

The values in the denominator of both the G color value and the alpha value scale the maximum contribution of each sphere so that if enough completely-filled spheres corresponding to the nominal depth of sample along the suggested pose (l_{D}) are within L_{N} mm of the virtual camera, the green channel will saturate. In theory, the choice of the green channel value and the alpha value for a sphere can could be made in any combination such that product of the two is

$$255 \cdot \frac{1.5 \cdot {f}_{i}}{{l}_{D}} .$$

However, the G and alpha values are chosen to be the largest values possible for each,

$$\sqrt{255 \cdot \frac{1.5 \cdot {f}_{i}}{{l}_{D}}} ,$$

to avoid hardware quantization errors.

After either of the planning methods are invoked, the next step in the bronchoscopic workflow is the generation of reports to convey the planning data to the physician. The next section details the reporting system component.

### EXAMPLE

We have conducted an MDCT image-analysis study to determine the efficacy and performance of a pose-orientation selection strategy in accordance with the present invention. In this study, we examined high-resolution MDCT chest scans of 11 patients with 20 diagnostic ROIs in the central chest region, defined at the direction of a physician, that were identified for potential follow-on transbronchial needle aspiration (TBNA) procedures. The purpose of the study was to verify the route-planning methods give appropriate routes to ROIs in a clinically-reasonable timeframe while accommodating realistic procedural, anatomical, and physical constraints.

Table I, below, summarizes the cases examined in this study. For most patients, ROIs were located along the trachea or in a subcarinal position at Mountain stations M4 (lower paratracheal) and M7 (inferior mediastinal), which are typically accessible for TBNA with modern videobronchoscopes [26]. Patients 20349.3.29, 20349.3.37, and 20349.3.37, however, were primarily enrolled for a peripheral pilot study [10]. These cases contained multiple ROIs, some of which were peripheral nodules and some of which were near lobar bronchi, and had the potential to be sampled via TBNA with a standard videobronchoscope.

TABLE I

| Patient | ROI | Location | ROI Axis Lengths (mm) | | |
|---|---|---|---|---|---|
| 20349.3.3 | 1 | Lower Paratracheal (M4) | 8.8 | 10.7 | 23.9 |
| | 2 | Lower Paratracheal (M4) | 10.4 | 13.8 | 17.5 |
| | 3 | Lower Paratracheal (M4) | 8.5 | 11.5 | 12.9 |
| | 4 | Lower Paratracheal (M4) | 9.2 | 10.6 | 15.4 |
| | 5 | Inferior Mediastinal (M7) | 13.3 | 19.2 | 33.5 |
| 20349.3.6 | 1 | Lower Paratracheal (M4) | 9.3 | 10.5 | 14.5 |
| 20349.3.7 | 1 | Lower Paratracheal (M4) | 8.8 | 10.6 | 12.9 |
| 20349.3.9 | 1 | Left Interlobar (M11) | 9.8 | 11.5 | 19.9 |
| | 2 | Left Lobar (M12) | 3.7 | 9.2 | 13.0 |
| | 3 | Inferior Mediastinal (M7) | 4.3 | 12.9 | 16.7 |
| | 4 | Hilar (M10) | 3.2 | 7.5 | 10.9 |
| 20349.3.11 | 2 | Lower Paratracheal (M4) | 13.1 | 17.0 | 34.0 |
| 20349.3.16 | 1 | Lower Paratracheal (M4) | 10.4 | 18.2 | 26.0 |
| | 2 | Inferior Mediastinal (M7) | 15.9 | 17.2 | 24.0 |
| 20349.3.29 | 1 | Left Lobar (M12) | 30.0 | 42.4 | 27.1 |
| 20349.3.37 | 1 | Right Interlobar | 9.6 | 14.4 | 19.9 |
| 20349.3.39 | 1 | Right Lobar (M12) | 12.6 | 13.6 | 15.4 |
| | 2 | Lower Paratracheal (M4) | 11.5 | 19.0 | 23.6 |
| 20349.3.40 | 1 | Lobar (M12) | 5.6 | 11.4 | 17.7 |
| 21405.13 | 1 | Inferior Mediastinal (M7) | 11.6 | 14.8 | 22.1 |

Table I shows description of cases examined in the evaluation of calculated pose orientations. Patients are identified according to the institutional review board protocol. ROI locations are given according to the Mountain system [26]. The ROI axes lengths correspond to the lengths of principal axes of the best-fit ellipsoid, found via principal component analysis [16].

All planning and visualization was conducted on a Dell Precision 380 workstation with 4 GB memory and a dual-core 3.46 GHz Intel processor. The GPU was an ATI Radeon X1900XTX 512 MB video card. The parameters used in the automated planning system were chosen to reflect modern bronchoscopic equipment. At our university's medical center, the smallest-diameter bronchoscope with a working channel large enough to accept a TBNA needle is the 4.9 mm diameter Olympus EVIS Exera II true color videobronchoscope. This bronchoscope model was chosen for our evaluation because of its small size relative to other bronchoscopes, which can have diameters >6 mm, allowing the EVIS Exera II to traverse a significant portion of the central-chest airways. In our planning system, we modeled the bronchoscope as 4.9 mm in diameter with a 10 mm rigid tip that was precluded from deviating from a candidate route destination's view site normal direction by >60°. The bronchoscopic accessory was modeled as a standard needle with rigid-tip length 20 mm.

To model the airway trees, we used the segmentation method described in Graham et al., the likelihood-image of the surfaces of Gibbs et al., the airway centerlines method of Yu et al., and the quantitative measurements Gibbs [9, 8, 7, 36, 6]. Because of the particular anatomic sensitivity of the pulmonary artery and aorta, we included these vessels in the obstacle-avoidance calculations of the planning strategy. To obtain the obstacle segmentations, we used a method described in Taeprasartsit and Higgins [33]. The safety envelope was defined by a FOV of 22.5° and a safety length of 22.5 mm. In the expected depth-of-sample approximation, we used a total of 22 DOS directions for each pose, with the directions comprising an expected depth of sample field of view of 15°.

To find the best routes to each ROI, we found up to 3 pose orientations with the highest expected depths of sample. The calculated routes were determined such that the view sites associated with each of the best pose orientations were separated by a topological view-site-to-view-site distance of ≧5 mm. That is, the total distance traveled along the centerlines from one of the best poses' view site location to any other pose's view site location in the solution set was required to be ≧5 mm. For this study we were interested in finding the best feasible routes, so we utilized the 100^{th} percentile expected depth of sample to score a candidate route destination. In addition to the expected depth of sample of the top poses, we also determined the nominal depth of sample along the pose direction. The nominal depth of sample scales the endoluminal blending intensity of the ROI targets to give a sense of the depth of sample of the ROI by the visualization approach described in detail herein.

Table II lists a summary of results in the evaluation of calculated pose orientations. All planning was conducted using the same set of previously described bronchoscopic and safety parameters. “Num. Routes Found” provides the number of feasible non-zero expected-depth-of-sample routes found for each ROI. For routes with at least one feasible route was found, the following columns describe the properties of the highest-scoring route. These columns give the expected depth of sample (EDOS), depth of sample (DOS) of the nominal pose orientation, distance from the pose location to the ROI center of mass, distance from the pose location to the ROI surface, and minimum inner diameter of the airways to the route destination. The Overall Time gives the running time required for the planning, including steps that need be performed only once per ROI. The Route-Specific Time gives the execution time required for the steps that are performed for each ROI each time the planning parameters (e.g., the safety FOV) are changed.

TABLE II

| | | Num. | | Nom. | Dist to | Dist to | Min. Route | Overall | Route-Specific |
|---|---|---|---|---|---|---|---|---|---|
| | | Routes | EDOS | DOS | ROI Cent | ROI Surf | Diam | Time | Time |
| Patient | ROI | Found | (mm) | (mm) | (mm) | (mm) | (mm) | (sec) | (sec) |
| 20349.3.3 | 1 | 3 | 9.4 | 12.8 | 13.4 | 6.0 | 12.7 | 35.8 | 12.6 |
| | 2 | 3 | 5.8 | 8.5 | 16.8 | 11.4 | 12.7 | 31.6 | 8.4 |
| | 3 | 1 | 6.5 | 10.0 | 9.8 | 15.6 | 12.7 | 30.7 | 8.1 |
| | 4 | 3 | 4.1 | 6.4 | 21.2 | 13.7 | 12.7 | 32.6 | 9.7 |
| | 5 | 3 | 14.9 | 16.6 | 13.8 | 2.2 | 13.3 | 42.1 | 16.4 |
| 20349.3.6 | 1 | 3 | 8.8 | 12.1 | 15.1 | 8.1 | 15.5 | 29.9 | 11.9 |
| 20349.3.7 | 1 | 3 | 7.9 | 12.2 | 14.4 | 7.4 | 10.9 | 24.3 | 7.3 |
| 20349.3.9 | 1 | 0 | — | — | — | — | — | 33.4 | 13.5 |
| | 2 | 1 | 6.3 | 9.7 | 10.2 | 3.8 | 8.9 | 30.6 | 11.9 |
| | 3 | 1 | 3.7 | 3.7 | 17.6 | 16.0 | 7.7 | 27.3 | 8.6 |
| | 4 | 3 | 5.8 | 10.2 | 11.0 | 6.5 | 9.7 | 27.8 | 9.2 |
| 20349.3.11 | 1 | 3 | 11.8 | 17.0 | 11.3 | 3.9 | 13.7 | 25.0 | 7.7 |
| 20349.3.16 | 1 | 3 | 10.1 | 12.3 | 12.7 | 6.5 | 15.3 | 91.5 | 62.1 |
| | 2 | 3 | 16.2 | 17.3 | 13.0 | 2.4 | 10.4 | 42.0 | 12.8 |
| 20349.3.29 | 1 | 0 | — | — | — | — | — | 83.3 | 42.9 |
| 20349.3.37 | 1 | 3 | 1.1 | 1.6 | 21.2 | 15.3 | 7.7 | 27.9 | 9.7 |
| 20349.3.39 | 1 | 3 | 14.0 | 16.0 | 15.0 | 2.7 | 8.4 | 72.7 | 44.8 |
| | 2 | 0 | — | — | — | — | — | 48.0 | 22.6 |
| 20349.3.40 | 1 | 0 | — | — | — | — | — | 33.4 | 12.5 |
| 21405.13 | 1 | 3 | 13.3 | 14.0 | 13.6 | 5.6 | 11.0 | 30.9 | 14.2 |
| Mean | | 2.2 | 8.4 | 11.0 | 14.9 | 7.9 | 10.7 | 40.0 | 17.3 |

Table II summarizes the results of the pose-orientation planning system. Of the 20 ROIs considered, feasible routes were determined to 16 of the ROIs. For those cases where feasible routes were found, the table provides the expected and nominal depth of sample at the suggested pose orientation. We also provide the distance to the ROI center-of-mass and the ROI surface and the minimum airway diameter encountered along the route to the ROI.

For all cases, we provide the computational time required for all pose-orientations steps (Overall Time) and the time required for those steps that must be performed each time a route to an ROI is planned if the bronchoscope, accessory, or safety envelope parameters change (Route-Specific Time). The Overall Time is the time required to plan a typical case, and this includes determining the ROI and obstacle surfaces, associating voxels with view sites, and all the pose orientation calculations for a given view site. The Route-Specific Time includes only the time spent on the pose-orientation calculations. These include the feasibility and expected depth of sample scoring computations. The average overall planning time, on a per ROI basis, was 40.0 sec, with a standard deviation of ±19.4 sec. These execution times fit well within the clinical workflow and allow for the computations to be run with different parameters, if desired, without the physician or technician wasting considerable time.

An examination of the routes in a virtual bronchoscopic system determined the viability of the 16 feasible routes. The 4.9 mm diameter, 10 mm-long rigid bronchoscope tip and the 20 mm needle was rendered within the airway surfaces and examined extraluminally. In this examination, we made sure that no obstacles were hit and the relative geometries between the airway, bronchoscope, and ROI were appropriate. We also examined endoluminal renderings of the routes to examine the new visualization strategy. These renderings were visually compared with the previous strategy of rendering solid ROIs without obstacle information to demonstrate the improved visualization approach.

In the cases where routes were not found, there were two modes of “failure.” In the first mode, the ROIs were located along narrow airways, typically deeper within the airway tree. Such ROI locations precluded the 4.9 mm bronchoscope from performing a safe TBNA within 20 mm of the ROT. This alerts the physician that such cases are unreasonable. Choosing a smaller bronchoscope model led to successful calculations for these ROIs, indicating that smaller equipment than what is currently available at our university's medical center could allow for these procedures.

In the second failure mode, the ROIs co-mingled with the major blood vessels. In these circumstances, no feasible routes could be found within the 22.5° FOV, 22.5 mm-long safety envelope. In these cases, the physicians have an increased likelihood of piercing the blood vessels during the procedure. If this risk is deemed tolerable, a smaller safety envelope could be used. For instance, we chose to model a more aggressive procedure with a narrow 10° FOV, 20 mm-long safety envelope. With these modifications, we were able to successfully plan routes to the remaining 4 ROIs. Because the calculations of the method are so fast, it is not unreasonable to re-run planning with multiple parameter sets to see if sampling a particular ROI is feasible with a smaller bronchoscope or with a less-conservative safety margin. The physician can then weigh the relative risk to benefit for the patient if a more aggressive parameter set is required to find a feasible route.

This disclosure has primarily focused on 3D bronchoscopic route planning. This is reflective of our research, where we are primarily concerned with the 3D airway tree, as depicted in 3D multidetector computed tomography (MDCT) images, with videobronchoscopy as the follow-on clinical method used for a variety of diagnostic procedures. The ROI regions we typically encounter are lymph nodes, suspect nodules, tumors, and infiltrates, but they may be any other anatomical target the physician may define. While the lungs and the airway tree, as represented by MDCT images, are our primary focus, the methods described can easily be extended. The volumetric image may be obtained by CT, MRI, or any other similar technique. The organ of interest could be the colon, coronary arterial tree, or any other like organ. As such, the examples and results given in this document illustrate a specific application of a general technique.

Features, aspects and variations of the systems and methods described herein may be combined and such combinations are specifically part of the present invention except where such combinations would be mutually exclusive or act to make the invention inoperable.

### REFERENCES

[1] F. Asano and Y. Matsuno and N. Shinagawa and K. Yamazaki and T. Suzuki and H. Moriya. A virtual bronchoscopic navigation system for pulmonary peripheral lesions. Chest, 130(2); 559-66, 2006.
[2] F. Asano and Y. Matsuno and A. Tsuzuku and M. Anzai and N. Shinagawa and K. Yamazaki and T. Ishida and H. Moriya. Diagnosis of peripheral pulmonary lesions using a bronchoscope insertion guidance system combined with endobronchial ultrasonography with a guide sheath. Lung Cancer, 60(3):366-373, 2008.
[3] M. Y. Dolina and D. C. Cornish and S. A. Merritt and L. Rai and R. Mahraj and W. E. Higgins and R. Bascom. Interbronchoscopist Variability in Endobronchial Path Selection: A Simulation Study. Chest, 133(4):897-905, 2008.
[4] Russell Gayle and Paul Segars and Ming C. Lin and Dinesh Manocha. Path Planning for Deformable Robots in Complex Environments. Robotics Science and Systems, 2005.
[5] B. Geiger and A. P. Kiraly and D. P. Naidich and C. L. Novak. Virtual bronchoscopy of peripheral nodules using arteries as surrogate pathways. In A. A. Amini and A. Manduca, editors, SPIE Medical Imaging 2005: Physiology, Function, and Structure from Medical Images, pages 352-360, 2005.
[6] J. D. Gibbs. Three Dimensional Route Planning for Medical Inage Reporting and Endoscopic Guidance. PhD thesis, The Pennsylvania State University, 2008.
[7] J. D. Gibbs and M. W. Graham and W. E. Higgins. 3D MDCT-Based System for Planning Peripheral Bronchoscopic Procedures. Comput Biol Med, 2008 (in review).
[8] J. D. Gibbs and M. W. Graham and K. C. Yu and W. E. Higgins. Integrated System for Planning Peripheral Bronchoscopic Procedures. In X. P. Hu and A. V. Clough, editors, SPIE Medical Imaging 2008: Physiology, Function, and Structure from Medical Images, pages 69160H-1-69160H-15, 2008.
[9] M. W. Graham and J. D. Gibbs and W. E. Higgins. A Robust System for Human Airway Tree Segmentation. In J. P. W. Pluim and J. M. Reinhardt, editors, SPIE Medical Imaging 2008: Image Processing, pages 69141J-1-69141J-18, 2008.
[10] M. W. Graham and J. D. Gibbs and K. C. Yu and D. C. Cornish and M. S. Khan and R. Bascom and W. E. Higgins. Image-Guided Bronchoscopy for Peripheral Nodule Biopsy: A Human Feasibility Study. Am J Respir Crit Care Med, pages A893, 2008.
[11] E. F. Haponik and S. L. Aquino and D. J. Vining. Virtual bronchoscopy. Clinics in Chest Med., 20(1):201-217, 1999.
[12] J. A. Hartigan and M. A. Wong. A K-Means Clustering Algorithm. Applied Statistics, 28:100-108, 1979.
[13] W. E. Higgins and J. D. Gibbs. Method and apparatus for 3D route planning and extension. 2008 (application submitted).
[14] A. Jemal and R. C. Tiwari and T. Murray and A. Ghafoor and A. Samuels and E. Ward and E. Feuer and M. Thun. Cancer Statistics, 2004. CA Cancer J. Clin., 54:8-29, 2004.
[15] Y. J. Jeong and C. A. Yi and K. S. Lee. Solitary pulmonary nodules: detection, characterization, and guidance for further diagnostic workup and treatment. AJR Am J Roentgenol, 188(1):57-68, 2007.
[16] I. T. Joliffe. Principal Component Analysis. Springer, 2nd edition, 2002.
[17] A. P. Kiraly and J. P. Helferty and E. A. Hoffman and G. McLennan and W. E. Higgins. 3D Path Planning for Virtual Bronchoscopy. IEEE Trans. Medical Imaging, 23(11):1365-1379, 2004.
[18] M. Kukuk. Modeling the internal and external constraints of a flexible endoscope for calculating its workspace: application in transbronchial needle aspiration guidance. SPIE Medical Imaging 2002: Visualization, Image-Guided Procedures, and Display, S. K. Mun (ed.), v. 4681:539-550, 2002.
[19] Murkus Kukuk. A Model-Based Approach to Intraoperative Guidance of Flexible Endoscopy. PhD thesis, University of Dortmund, 2002.
[20] W. E. Lorensen and H. E. Cline. Marching Cubes: A high resolution 3D surface construction algorithm. Computer Graphics, 21(4); 163-169, 1987.
[21] H. P. McAdams and P. C. Goodman and P. Kussin. Virtual bronchoscopy for directing transbronchial needle aspiration of hilar and mediastinal lymph nodes: a pilot study. AJR Am J Roentgenol, 170(5):1361-1364, 1998.
[22] S. A. Merritt and J. D. Gibbs and K. C. Yu and V. Patel and L. Rai and D. C. Cornish and R. Bascom and W. E. Higgins. Real-Time Image-Guided Bronchoscopy for Peripheral Lung Lesions: A Phantom Study. Chest, 2008 (in press).
[23] S. A. Merritt and L. Rai and J. D. Gibbs and K. Yu and W. E. Higgins. Method for continuous guidance of endoscopy. In A. Manduca and X. P. Hu, editors, SPIE Medical Imaging 2007: Physiology, Function, and Structure from Medical Images, pages 65110O-1-65110O-12, 2007.
[24] S. A. Merritt and L. Rai and W. E. Higgins. Real-time CT-video registration for continuous endoscopic guidance. In A. Manduca and A. A. Amini, editors, SPIE Medical Imaging 2006: Physiology, Function, and Structure from Medical Images, pages 370-384, 2006.
[25] H Minami and Y Ando and F Nomura and S Sakai and K Shimokata. Interbronchoscopist variability in the diagnosis of lung cancer by flexible bronchoscopy. Chest, 105(2):1658-1662, 1994.
[26] C. Mountain and C. Dresler. Regional lymph node classification for lung cancer staging. Chest, 111(6):1718-1723, 1997.
[27] Matt Pharr and Greg Humphreys. Physically Based Rendering: From Theory to Implementation. Morgan Kaufmann, 2004.
[28] M. P. Rivera and F. Detterbeck and A. C. Mehta. Diagnosis of Lung Cancer: The Guidelines. Chest, 123:129 S-136S, 2003.
[29] W. Schroeder and K. Martin and B. Lorensen. The Visualization Toolkit, 2nd. Ed. Prentice Hall, Upper Saddle River, N.J., 1998.
[30] N. Shinagawa and K. Yamazaki and Y. Onodera and F. Asano and T. Ishida and H. Moriya and M. Nishimura. Virtual bronchoscopic navigation system shortens the examination time—Feasibility study of virtual bronchoscopic navigation system. Lung Cancer, 56(2):201-206, 2007.
[31] Shinagawa, Naofumi and Yamazaki, Koichi and Onodera, Yuya and Asahina, Hajime and Kikuchi, Eiki and Asano, Fumihiro and Miyasaka, Kazuo and Nishimura, Masaharu. Factors Related to Diagnostic Sensitivity Using an Ultrathin Bronchoscope Under CT Guidance. Chest, 131(2):549-553, 2007.
[32] A. D. Sihoe and A. P. Yim, Lung cancer staging. J Surgical Research, 117(1):92-106, 2004.
[33] Pinyo Taeprasartsit and William E. Higgins. Method for extracting the aorta from 3D CT images. SPIE Medical Imaging 2007. Image Processing, 6512(1):65120J, 2007.
[34] K. P. Wang and A. Mehta, eds. Flexible Bronchoscopy. Blackwell Science, Cambridge, Mass., 1995.
[35] R. S. Wright, Jr., and B. Lipchak and Nicholas Haemel. OpenGL Super Bible, 4th. Ed. Addison Wesley, 2007.
[36] K, C, Yu and E. L. Ritman and W. E. Higgins. System for the Analysis and Visualization of Large 3D Anatomical Trees. Comput Biol Med, 37(12):1802-18207, 2007.

All of the above referenced patents, patent applications and publications are hereby incorporated by reference in their entirety.

We claim:

1. A method of planning a route for an endoscope through an anatomical luminal structure to a target, the endoscope including a working lumen with a working end, the method comprising the steps of:
generating a 3D model of a luminal structure including a target;
calculating a plurality of candidate poses along the luminal structure, each candidate pose indicating the location and direction of the working end of the endoscope in relation to the target; and
planning the route as a function of which candidate pose or poses would optimize the sampling of the target.

2. The method of claim 1, wherein the sampling optimization is based upon a physical characteristic of the target.

3. The method of claim 2, wherein:
the physical characteristic is the volume of the target; and
the optimization corresponds to the candidate pose representing the largest volume of target sample.

4. The method of claim 1, further comprising the steps of:
deriving one or more obstacles or constraints from endoscope information or anatomical information; and
eliminating one or more of the candidate poses as a function of the obstacles or constraints.

5. The method of claim 4, wherein the endoscope information includes endoscope diameter.

6. The method of claim 4, wherein the anatomical information includes the diameter of the luminal structure.

7. The method of claim 4, wherein the anatomical information includes the location of a blood vessel.

8. The method of claim 1, including the steps of:
obtaining endoscope flexibility information; and
eliminating one or more of candidate poses in accordance with the flexibility information.

9. The method of claim 1, further comprising the step of generating a report including at least one candidate pose.

10. The method of claim 1, wherein;
the luminal structure is an airway tree; and
the endoscope is a bronchoscope.

11. The method of claim 1, further comprising the step of indicating at least one of the poses.

12. The method of claim 11, wherein the step of indicating is carried out by displaying an icon comprising a direction and location.

13. The method of claim 12, wherein the icon has an arrow shape.

14. The method of claim 12, wherein:
the target is displayed in combination with the icon; and
the target includes a pose-dependant visual effect.

15. The method of claim 1, further including the step of ensuring that the working end of the endoscope fits within the luminal structure at a final location.

16. The method of claim 1, further including the step of obtaining information about an appliance to be used in combination with the endoscope.

17. The method of claim 16, wherein the appliance information is received in the form of an appliance model number.

18. The method of claim 16, wherein the optimization of target sampling takes into account the appliance information.

19. The method of claim 16, wherein the appliance is appliance is a needle, coring needle, brush, forceps, RF ablator, cryo-ablator, oxygen sensor, electrical sensor, implant delivery catheter, aspiration device, fluid delivery device, or temperature sensor.

20. The method of claim 16, wherein the appliance information comprises a conic volume originating from the working end of the endoscope.

21. The method of claim 16, wherein the appliance is configured to deliver an implant.

22. The method of claim 21, wherein the implant is a conduit, valve, balloon, or plug.

23. The method of claim 4, further including the step of receiving endoscope information in the form of an endoscope model number.

24. A method of guiding a surgical appliance towards a region of interest (ROI) prior to or during an endoscopic procedure, comprising the steps of:
displaying a region of interest (ROI) to be sampled and a rendered lumen in a vicinity of the ROI on a display;
generating an instant pose corresponding to a position and direction of a working end of an endoscope including a surgical appliance;
indicating, on the display, a visual effect derived from a physical dimension of the ROI as a function of the instant pose and the surgical appliance; and
adjusting the position or direction of the working end and observing a change in the visual effect on the display.

25. The method of claim 24, wherein:
the pose corresponds to an endoscope in use during a live procedure; and
the instant pose is generated as the working end of the manipulated toward the ROI.

26. The method of claim 24, further comprising the step of selecting the instant pose corresponding to a maximum change in visual effect.

27. The method of claim 24, wherein a pose icon representative of the instant pose is displayed on the display.

28. The method of claim 26, wherein the visual effect is a change in the color intensity of the ROI on the display.

29. The method of claim 28, wherein the color intensity increases if the physical dimension of the ROI to be sampled increases.

30. The method of claim 29, wherein the physical dimension is the depth of a tissue sample.

31. The method of claim 24, further including the step of registering a pre-computed virtual image of the ROI and lumen at the instant pose with a real image arising from an endoscopic device in a position corresponding to the instant pose.

32. The method of claim 24, wherein the ROI is a lymph node, tumor, or lesion located within a lung.

33. The method of claim 24, wherein the endoscope is a virtual endoscope controlled with an input device.

34. A system for planning or visualizing a route through a branched lumen to a target region of interest (ROI), comprising:
a memory storing a reconstructed 3D model of the branched lumen and the ROI;
a processor including a route module operative to compute a route to the ROI and a pose module operative to compute a pose based on a physical dimension of a tissue sample obtained from the ROI using an appliance to obtain the sample.

35. The system of claim 34, wherein the pose is computed in real time based on a position and direction of the working end of an endoscope.

36. The system of claim 34, further including a display for displaying a visual effect corresponding to the pose.

37. The system of claim 36, wherein:
the physical dimension is the depth of sample; and
the visual effect is color intensity of an ROI image, the color intensity increasing as the depth of sample increases.

38. The system of claim 34, wherein the pose is a candidate pose based on a simulated endoscope position and direction.

39. The system of claim 34, wherein the pose is derived from a plurality of candidate poses.

40. The system of claim 34 wherein the processor is further operative to derive information and to eliminate one or more poses based on said information, wherein said information is at least one of obstacle information, appliance information, and anatomical information.

41. The system of claim 40 wherein said processor is further operative to indicate one of the following 1) a first route and first pose corresponding to a route and pose that provides the sample with a maximum physical dimension, and 2) the absence of a first route or first pose based on said information.
